(19) United States
(12) Patent Application Publication
(10) Pub. No.: US 2023/0333095 A1
(43) Pub. Date: Oct. 19, 2023

(54) METHOD FOR DETECTING AN ANALYTE OF INTEREST IN A SAMPLE

(71) Applicant: Roche Diagnostics Operations, Inc., Indianapolis, IN (US)

(72) Inventors: Maksim Fomin, Bergisch Gladbach (DE); Dieter Heindl, Muenchen (DE); Lars Hillringhaus, Königsdorf-Schönrain (DE); Hannes Kuchelmeister, München (DE); Mohamed Yosry Hassan Mohamed, München (DE); Tobias Oelschlaegel, Geretsried-Gelting (DE); Sona Simonyiova, Muenchen (DE)

(21) Appl. No.: 18/339,799

(22) Filed: Jun. 22, 2023

Related U.S. Application Data
(63) Continuation of application No. PCT/EP2021/086724, filed on Dec. 20, 2021.

(30) Foreign Application Priority Data
Dec. 22, 2020 (EP) 20216267.3

(51) Int. Cl. G01N 33/533; G01N 33/68; C07F 15/02

(52) U.S. Cl. CPC G01N 33/533; G01N 33/6887; C07F 15/025; G01N 2800/325

(57) ABSTRACT

The present invention relates to a method for determining at least one analyte of interest. The present invention further relates to a kit, a complex, a method to synthesize a complex and the use thereof for detecting the analyte of interest in the sample.

### FIELD OF THE INVENTION

The present invention relates to a method for determining at least one analyte of interest. The present invention further relates to a kit, a complex, a method to synthesize a complex and the use thereof for detecting the analyte of interest in the sample.

### BACKGROUND OF THE INVENTION

U.S. Pat. No. 9,511,150 reports sugar alcohols and crosslinking reagents, macromolecules, and therapeutic bioconjugates. US 2016/0250896 report phosphonates and sulfonates and hydrophilic linkers, and uses of such linkers for conjugation of drugs to cell binding molecules. US 2010/0009902 reports conjugation with PEG (polyethylene glycol)having a selected molecular weight. Vlahov I. R. et al J. Org. Chem. 75 (2010) 3685-3691 report a carbohydrate-based synthetic approach to control toxicity profiles of folate-drug conjugates. In more detail, the document discloses incorporation of 1-amino-1-deoxy-d-glucitol-γ-glutamate subunits into a peptidic backbone. Synthesis of Fmoc-3,4; 5,6-di-O-isopropylidene-1-amino-1-deoxy-d-glucitol-γ-glutarnate, suitable for Fmoc-strategy solid-phase peptide synthesis (SPPS), was achieved in four steps from δ-gluconolactone. Addition of alternating glutamic acid and 3,4; 5,6-di-O-isopropylidene-1-amino-1-deoxy-d-glucitol-γ-glutamate moieties onto a cysteine-loaded resin, followed by the addition of folate, deprotection, and cleavage, resulted in the isolation of the new folate-spacer: Pte-γGlu-(Glu(1-amino-1-deoxy-d-glucitol)-Glu)₂-Glu(1-amino-1-deoxy-d-glucitol)-Cys-OH.

A particular technical feature known to the art of polymer chemistry is poly dispersity which denotes the lack of uniformity in the amount of incorporated monomers and/or polymer chain length. A particular technical problem is posed by frequently observed polydispersity of linker-comprised compounds and conjugates.

Specifically, PEG-based linkers may have such drawbacks. Owing to the technical features of typically used polymerization chemistry, resulting high-molecular-weight PEG molecules are characterized by substantial polydispersity. I.e. a typical polymerization yields a mixture of molecules with different molecular masses. Using such mixed-molecular-weight PEG molecules as linkers leads to a propagation of the polydispersity among the resulting conjugates. As a result, any analysis of the conjugates is complicated as a desired conjugate would be defined a uniform molecular weight. Such uniform molecular weight is however not attained. In addition, despite the hydrophilicity of a PEG moiety in a spacer, certain conjugates with PEG still lack sufficient solubility.

Polysaccharides also tend to be polydisperse and variable in structure, due to the complexity and difficulties of sugar chemistry. Synthesis of longer and more complex alcohols requires elaborate and low yielding protecting group manipulations.

There is thus an urgent need in the art to overcome the above mentioned problems.

For the present invention, specific substantially monodisperse linker molecules with polyols have been devised which can be used to advantageously crosslink functional molecules. The inventors have found that certain linker molecules with polyols not only offer superior hydrophilicity over PEG-containing derivatives. In an exemplary setting a complex comprising such a linker which crosslinks an analyte-specific binding agent and a label compound produces an improved signal-to-noise ratio in an analyte detection assay. Further, the linker and/or complex shows monodispersity, which preferably results from the peptide synthesis and can be shown by the HPLC chromatograms.

It is an object of the present invention to provide a method for detecting an analyte of interest in a sample. Further, it is an object of the present invention to provide a a kit, a complex, a method to synthesize a complex and the use thereof for detecting the analyte of interest in the sample.

This object is or these objects are solved by the subject matter of the independent claims. Further embodiments are subjected to the dependent claims.

### SUMMARY OF THE INVENTION

In the following, the present invention relates to the following aspects:

In a first aspect, the present invention relates to a method for detecting an analyte of interest in a sample comprising the steps of
a) Providing the sample comprising the analyte of interest,
b) Providing a complex comprising a linker, wherein the linker covalently binds a label compound and an analyte-specific binding agent, wherein the label compound is capable of producing a detectable signal, preferably a chemiluminescence based signal,
c) Coupling the sample of step a) with the complex of step b),
d) Detecting the analyte of interest by using the detectable signal of the label compound,
wherein the complex is a compound of formula I:

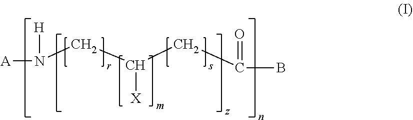

wherein A represents the label compound and B represents the analyte-specific binding agent or vice versa,
X is OH or (CHOH)ₜ—CH₂OH with t≥1, preferably t=1, 3, 5 or 7,
m is an integer from 1 to 8, preferably ≥2, in particular 2 to 8,
n is an integer from 2 to 20, preferably 5 to 20,
r is an integer and ≥0, preferably 0, wherein r≥1 in case X=OH,
s is an integer and ≥0, preferably 0, and
z is an integer and ≥1.

In a second aspect, the present invention relates to the use of the method according to the first aspect of the present invention for detecting the analyte of interest in the sample.

In a third aspect, the present invention relates to a kit for performing detection of an analyte of interest in a sample, the kit comprising in separate containers
a) a solid phase capable of immobilizing the analyte;
b) a compound of formula I:

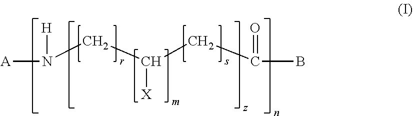

wherein A represents the label compound and B represents the analyte-specific binding agent or vice versa,
X is OH or (CHOH)ₜ—CH₂OH with t≥1, preferably t=1, 3, 5 or 7,
n is an integer from 1 to 8, preferably ≥2, in particular 2 to 8,
n is an integer from 2 to 20, preferably 5 to 20,
r is an integer and ≥0, preferably 0, wherein r≥1 in case X=OH,
s is an integer and ≥0, preferably 0, and
z is an integer and ≥1.

In a fourth aspect, the present invention relates to the use of the kit according to the third aspect of the present invention for detecting the analyte of interest in the sample.

In a fifth aspect, the present invention relates a complex of formula I.

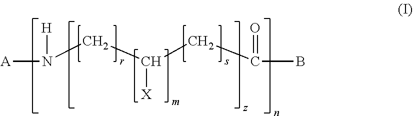

wherein A represents the label compound and B represents the analyte-specific binding agent or vice versa,
X is OH or (CHOH)ₜ—CH₂OH with t≥1, preferably t=1, 3, 5 or 7,
M is an integer from 1 to 8, preferably ≥2, in particular 2 to 8,
n is an integer from 2 to 20, preferably 5 to 20.
r is an integer and ≥0, preferably 0, wherein r≥1 in case X=OH,
s is an integer and ≥0, preferably 0, and
z is an integer and ≥1.
   preferably wherein the compound is suitable to detect an analyte of interest in a sample.

In a sixth aspect, the present invention relates to a method to synthesize a complex of the fifth aspect of the present invention comprising the steps of
a) Providing a monomer or derivatives thereof, wherein the monomer is an amino acid comprising an amino group, a carboxy group and at least one hydroxyl group, wherein the amino group or the carboxy group is protected by a first protecting group, and the at least one or each hydroxyl group is protected by a second protecting group,
b) Using the monomer in a process of solid phase peptide synthesis, cleaving the first and second protection group and forming a complex of formula III,

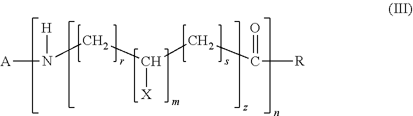

wherein A represents the label compound and R represents a second spacer or vice versa, wherein R is capable of covalently bonding to an analyte-specific binding agent or is covalently bonded to an analyte-specific binding agent,
X is OH or (CHOH)ₜ—CH₂OH with t≥1, preferably t=1, 3, 5 or 7,
m is an integer from 1 to 8, preferably ≥2, in particular 2 to 8,
n is an integer from 2 to 20, preferably 5 to 20,
r is an integer and ≥0, preferably 0, wherein r≥1 in case X=OH,
s is an integer and ≥0, preferably 0, and
z is an integer and ≥1.

### LIST OF FIGURES

FIG. 1 shows the Elecsys ECL technology.

FIGS. 2 to 4 show results of the Elecsys E170: Troponin T hs Assay according to the present invention and according to comparative examples.

### DETAILED DESCRIPTION OF THE INVENTION

Before the present invention is described in detail below, it is to be understood that this invention is not limited to the particular embodiments and examples described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Percentages, concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “4% to 20%” should be interpreted to include not only the explicitly recited values of 4% to 20%, but to also include individual values and sub-ranges within the indicated range. Thus, included in this numerical range are individual values such as 4, 5, 6, 7, 8, 9, 10, . . . 18, 19, 20% and sub-ranges such as from 4-10%, 5-15%, 10-20%, etc. This same principle applies to ranges reciting minimal or maximal values. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “detecting” the analyte of interest, as used herein refers to the quantification or qualification of the analyte of interest, e.g. the presence or amount of the analyte of interest in the sample, employing appropriate methods of detection described elsewhere herein.

In the context of the present disclosure, the term “analyte”, “analyte molecule”, or “analyte(s) of interest” are used interchangeably referring the chemical specis to be analysed via a detectable label. Chemical specis suitable to be analysed via a detectable label, i.e. analytes, can be any kind of molecule present in a living organism, include but are not limited to nucleic acid (e.g. DNA, mRNA, miRNA, rRNA etc.), amino acids, peptides, proteins (e.g. cell surface receptor, cytosolic protein etc.), drug molecules, metabolite or hormones (e.g. testosterone, estrogen, estradiol, etc.), fatty acids, lipids, carbohydrates, steroids, ketosteroids, secosteroids (e.g. Vitamin D), molecules characteristic of a certain modification of another molecule (e.g. sugar moieties or phosphoryl residues on proteins, methyl-residues on genomic DNA) or a substance that has been internalized by the organism (e.g. therapeutic drugs, drugs of abuse, toxin, etc.) or a metabolite of such a substance. Such analyte may serve as a biomarker. In the context of present invention, the term “biomarker” refers to a substance within a biological system that is used as an indicator of a biological state of said system. An “analyte” can be any molecule which can be bound by an analyte-specific receptor. In an embodiment, an analyte is an antigen of an infectious agent. Examples of infectious agents are viruses, bacteria and protozoic pathogens that infect humans. In an embodiment, an analyte is a viral antigen, in an embodiment a hepatitis virus antigen or a human retroviral antigen. In an embodiment, an analyte is a hepatitis C virus or hepatitis B virus or HIV antigen.

Generally, the term “receptor” denotes any compound or composition capable of recognizing a particular spatial and polar organization of a target molecule i.e. an epitopic site of an analyte. Thus, the term “analyte-specific receptor” as referred to herein includes analyte-specific reactants capable of binding to or complexing an analyte. This includes but is not limited to antibodies, specifically monoclonal antibodies or antibody fragments. Such a receptor can act as a catcher of the analyte, e.g. to immobilize the analyte. An epitope recognized by the antibody is bound, followed by labeled antibodies specific to another epitope of the analyte. Other receptors are known to those of skill in the art. The particular use of various receptors in a receptor-based analyte assay will be understood by those of skill in the art with reference to this disclosure.

Analytes or an analyte of interest may be present in a sample, e.g. in a biological or clinical sample. The term “biological or clinical sample” are used interchangeably herein, referring to a part or piece of a tissue, organ or individual, typically being smaller than such tissue, organ or individual, intended to represent the whole of the tissue, organ or individual. Upon analysis a biological or clinical sample provides information about the tissue status or the health or diseased status of an organ or individual. Examples of biological or clinical samples include but are not limited to fluid samples such as blood, serum, plasma, synovial fluid, spinal fluid, urine, saliva, and lymphatic fluid, or solid biological or clinical samples such as dried blood spots and tissue extracts. Further examples of biological or clinical samples are cell cultures or tissue cultures.

In context of the present disclosure, the term “antibody” relates to fill immunoglobulin molecules, specifically IgMs, IgDs, IgEs, IgAs or IgGs, as well as to parts of such immunoglobulin molecules, like Fab-fragments or V_{L}-, V_{H}- or CDR-regions. Furthermore, the term relates to modified and/or altered antibody, like chimeric and humanized antibodies. The term also relates to modified or altered monoclonal or polyclonal antibodies as well as to recombinantly or synthetically generated/synthesized antibodies. The term also relates to intact antibodies as well as to antibody fragments/parts thereof, like, separated light and heavy chains, Fab, Fab/c, Fv, Fab′, F(ab′)₂. The term “antibody” also comprises antibody derivatives, bifunctional antibodies and antibody constructs, like single chain Fvs (scFv), bispecific scFvs or antibody-fusion proteins.

In chemistry, “solid-phase synthesis” is a method in which molecules are covalently bound on a solid support material and synthesised step-by-step in a single reaction vessel utilising selective protecting group chemistry. As a specific embodiment, solid phase peptide synthesis is a common technique involving discrete steps for the synthesis of peptides. This approach permits unreacted reagents to be removed by washing without loss of product. Usually, peptides are synthesised from the carbonyl group side (C-terminus) to amino group side (N-terminus) of the amino acid chain. In peptide synthesis, an amino-protected amino acid is bound to a solid phase material such as, but not limited to, polystyrene beads, thereby forming a covalent bond between the carbonyl group and the resin, most often an amido or an ester bond. Then the amino group is deprotected and reacted with the carbonyl group of the next amino-protected amino acid. The solid phase now bears a dipeptide. This cycle is repeated to form the desired peptide chain. After all reactions are complete, the synthesised peptide is cleaved from the solid phase.

More specifically, the carboxyl moiety of each incoming amino acid is activated by one of several strategies and couples with the α-amino group of the preceding amino acid. The α-amino group of the incoming residue is temporarily blocked in order to prohibit peptide bond formation at this site. The residue is de-blocked at the beginning of the next synthesis cycle. In addition, reactive side chains on the amino acids are modified with appropriate protecting groups. The peptide chain is extended by reiteration of the synthesis cycle. Excess reagents are used to drive reactions as close to completion as possible.

The “blocking group” or “protecting group” or “protection group” used for blocking the α-amino group determines both the synthesis chemistry employed and the nature of the side-chain protecting groups. The two most commonly used α-amino protecting groups are Fmoc (9-fluorenyl-methoxy-carbonyl) and tBoc (tert.-butyloxycarbonyl). Fmoc side-chain protection is typically provided by ester, ether and urethane derivatives of tert.-butanol, while the typical corresponding tBoc protecting groups are ester, ether, and urethane derivatives of benzyl alcohol. The latter are usually modified by the introduction of electron-withdrawing halogens for greater acid-stability. Ether and ester derivatives of cyclopentyl or cyclohexyl alcohol are also employed.

After fully assembling the peptide the side-chain protecting groups are removed, if so desired, and the peptide is cleaved from the solid support, using conditions that inflict minimal damage on labile residues.

The product can be analyzed to verify the sequence thereafter. A synthetic peptide is usually purified by gel chromatography or HPLC.

Coupling of a label and/or a target molecule to the peptide are possible by different methods. As a non-limiting example, a building block amenable to SPPS may be incorporated into the peptide, wherein the building block comprises a reactive group which is optionally protected and which can be used for forming a linkage with a further compound of choice after the SPPS process. Alternatively, the compound of choice may already be attached to the building block when it enters the SPPS process. Other alternatives are possible.

The Fmoc protecting group is base-labile. It is usually removed with a dilute base such as piperidine. The side-chain protecting groups are removed by treatment with trifluoroacetic acid (TFA), which also cleaves the bond anchoring the peptide to the support. The tBoc protecting group is removed with a mild acid (usually dilute TFA). Hydrofluoric acid (HF) can be used both to deprotect the amino acid side chains and to cleave the peptide from the resin support. Fmoc is a gentler method than tBoc since the peptide chain is not subjected to acid at each cycle and has become the major method employed in commercial automated peptide synthesis.

The protecting groups for the amino groups mostly used in the peptide synthesis are 9-fluorenylmethyloxycarbonyl group (Fmoc) and t-butyloxycarbonyl (Boc). A number of amino acids bear functional groups in the side chain which must be protected specifically from reacting with the incoming N-protected amino acids. In contrast to Boc and Fmoc groups, these have to be stable over the course of peptide synthesis although they are also removed during the final deprotection of peptides.

A “label compound” includes a moiety that is detectable or that can be rendered detectable. The skilled person knows a label as a compound or composition capable of providing a detectable signal in conjunction with physical activation (or excitation) or chemical reagents and capable of being modified, so that the particular signal is diminished or increased.

Specific embodiments of a label compound label compound, which is capable of producing a detectable signal, include labels which are detectable by a number of commercially available instruments that utilize chemiluminescence, preferably electrochemiluminescence (ECL) for analytical measurements. Species that can be induced to emit ECL (ECL-active species) have been used as ECL labels. Examples of ECL labels include: i) organometallic compounds where the metal is from, for example, the noble metals of group VIII, including Ru-containing, Ir-containing and/or Os-containing organometallic compounds such as the tris-bipyridyl-ruthenium (RuBpy) moiety and ii) luminol and related compounds. Species that participate with the ECL label in the ECL process are referred to herein as ECL coreactants. Commonly used coreactants include tertiary amines (e.g., see U.S. Pat. No. 5,846,485), oxalate, and persulfate for ECL from RuBpy and hydrogen peroxide for ECL from luminol (see, e.g., U.S. Pat. No. 5,240,863. The light generated by ECL labels can be used as a reporter signal in diagnostic procedures (Bard et al., US5,238,808). For instance, an ECL label can be covalently coupled to a binding agent such as an antibody, nucleic acid probe, receptor or ligand; the participation of the binding reagent in a binding interaction can be monitored by measuring ECL emitted from the ECL label. Alternatively, the ECL signal from an ECL-active compound may be indicative of the chemical environment (see, e.g., U.S. Pat. No. 5,641,623 which describes ECL assays that monitor the formation or destruction of ECL coreactants). For more background on ECL, ECL labels, ECL assays and instrumentation for conducting ECL assays see U.S. Pat. Nos. 5,093,268; 5,147,806; 5,324,457; 5,591,581; 5,597,910; 5,641,623; 5,643,713; 5,679,519; 5,705,402; 5,846,485; 5,866,434; 5,786,141; 5,731,147; 6,066,448; 6,136,268; 5,776,672; 5,308,754; 5,240,863; 6,207,369 and U.S. Pat. No. 5,589,136; and WO99/63347, WO00/03233, WO99/58962, WO99/32662, WO99/14599, WO98/12539, WO97/36931 and WO98/57154.

The term “chemiluminescence based signal” refers to a signal that is produced by the emission of light (luminescence), as the result of a chemical reaction. This signal is detectable, e.g. by a number of commercially available instruments that utilize chemiluminescence.

In the context of the present disclosure, the term “complex” refers to the product produced by the reaction of a linker, a label compound and an analyte-specific binding agent. This reaction leads to the formation of a covalent bond between the label compound and the linker on the one hand and the linker and the analyte-specific binding agent on the other hand.

The term “linker” can refer to a compound serving as a spacer between the label compound and the analyte-specific binding agent and/or influencing the physico-chemical properties of the complex such as hydrophilicity and solubility.

The term “analyte-specific binding agent” refers to a (macro)molecule (protein, peptide, nucleic acid, etc.) capable of specifically binding the analyte of interest, e.g. a monoclonal antibody.

The term “A represents the label compound and B represents the analyte-specific binding agent or vice versa” means that A of formula I represents the label compound and B of formula I represents the analyte-specific binding agent. Alternatively, it means that that B of formula I represents the label compound and A of formula I represents the analyte-specific binding agent.

The term “Coupling the sample of step a) with the complex of step b)” refers to the reaction of the sample comprising or containing the analyte of interest with the complex of step b). Preferably, coupling refers to a covalently binding between the sample, preferably the analyte of interest, and the complex.

The term “peptide” means a molecule that is formed using naturally occurring L-amino acids or analogs thereof, like D-amino acids or N-alkylated amino acids or the like, Preferred amino acids are selected from the group consisting of Ala, Arg, Asn, Asp, Cys, Glu, Gin, Gly, His, Hyl, Hyp, Ile, Leu, Lys, Met, Phe, Pro, Ser, Thr, Trp, Tyr, and Val. Also other building blocks are possible having a carboxylic acid and an amino group. Additionally, modifications like fluorescence dyes or biotin are possible.

“Solid phase peptide synthesis (SPPS)” is a well established method. Merrifield et al. were the first who developed a convenient strategy for the build up of peptides by subsequently coupling amino acid monomers using a solid phase resin as a heterogeneous reaction medium (R. B. Merrifield, J. Am. Chem. Soc. 85 (1963) 2149-2154).

As a major advantage in comparison with the in-solution synthesis of peptides SPPS can be automated easily and impurities or by-products, reagents as well as unreacted starting material can be washed away while the product or intermediate remains tethered on the solid phase.

Normally the abovementioned Merrifield method starts with the attachment of the first C-terminal amino acid to a so called “linker” of a crosslinked polystyrene resin. The “linker” serves as a bridging element between the resin and the C-terminal amino acid of the peptide to be synthesized and the linker contains an acid sensitive bond to be used for the detachment of the peptide after synthesis.

As an example for a typical SPPS protocol the N-terminus can be protected with the 9-fluorenylmethoxycarbonyl (Fmoc) group, which is stable in acid, but removable by base. Any side chain functional groups are protected with base stable groups to make sure that only the N-terminal amino group incorporated in the peptide backbone can react-after removal of the Fmoc group-with the carboxylic acid group of the subsequent amino acid. As already mentioned the first step after the immobilization of the first amino acid is the deprotection of the amino function by removal of the Fmoc group using 20% piperidine in N,N-dimethylformamide (DMF). The amino function is coupled with an activated carboxylic acid via O-(Benzotriazol-1-yl)-N,N,N′,N′-tetramethyluronium hexafluorphosphate (HBTU) ester of the next amino acid in the presence of a base to form a new amide bond. This process is repeated until the desired peptide is assembled at the resin. As a last step the complete peptide is cleaved from the resin using a solution containing trifluoroacetic acid (TFA). The released peptide in the solution can be precipitated and washed before further purification.

This “classic” method for SPPS was optimized in recent years using modified resins, linkers, protective groups, coupling chemistries and cleavage procedures but the principle remains the same.

The term “solid phase” as used herein refers to a wide variety of materials including solids, semi-solids, gels, films, membranes, meshes, felts, composites, particles, resins, papers and the like typically used by those of skill in the art to sequester molecules. The solid phase can be a material, e.g. in a chromatographic column or as a specific embodiment a functionalized resin in a column of a device for solid phase synthesis. The solid phase can be non-porous or porous. The solid phase can be non-magnetic or magnetic (encompassing diamagnetic, paramagnetic, and superparamagnetic features).

Surfaces of solid phases as those described above may be modified to provide linkage sites, for example by bromoacetylation, silation, addition of amino groups using nitric acid, and attachment of intermediary proteins, dendrimers and/or star polymers. This list is not meant to be limiting, and any method known to those of skill in the art may be employed.

The term “polyol-units” refers to monomers (e.g. amino acids) comprising 1 or more OH groups. Such monomers can be covalently linked to each other to form a homo- or heteropolymer.

The term “linear linker” refers to a linker formed by polyol units where all or at least all the OH groups are directly bound to the linker backbone main chain.

The term “branched linker” refers to a linker formed by polyol units wherein one or more OH groups are bound to the side chain. The term “integer” means a whole number and not a fraction.

A “kit” is any manufacture (e.g. a package or container) comprising at least one reagent, e.g., a medicament for treatment of a disorder, or a probe for specifically detecting a biomarker gene or protein of the invention. The kit is preferably promoted, distributed, or sold as a unit for performing the methods of the present invention. Typically, a kit may further comprise carrier means being compartmentalized to receive in close confinement one or more container means such as vials, tubes, and the like. In particular, each of the container means comprises one of the separate elements to be used in the method of the first aspect. Kits may further comprise one or more other reagents including but not limited to reaction catalyst. Kits may further comprise one or more other containers comprising further materials including but not limited to buffers, diluents, filters, needles, syringes, and package inserts with instructions for use. A label may be present on the container to indicate that the composition is used for a specific application, and may also indicate directions for either in vivo or in vitro use. The computer program code may be provided on a data storage medium or device such as a optical storage medium (e.g., a Compact Disc) or directly on a computer or data processing device. Moreover, the kit may, comprise standard amounts for the biomarkers as described elsewhere herein for calibration purposes.

In this detailed description, references to “one embodiment”, “an embodiment”, or “in embodiments” mean that the feature being referred to is included in at least one embodiment of the technology with regards to all its aspects according to present disclosure. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the technology in all its aspects according to present disclosure can include any variety of combinations and/or integrations of the embodiments described herein.

### EMBODIMENTS

In a first aspect, the present invention relates to a method for detecting an analyte of interest in a sample comprising the steps of
a) Providing the sample comprising the analyte of interest,
b) Providing a complex comprising a linker, wherein the linker covalently binds a label compound and an analyte-specific binding agent, wherein the label compound is capable of producing a detectable signal, preferably a chemiluminescence based signal,
c) Coupling the sample of step a) with the complex of step b),
d) Detecting the analyte of interest by using the detectable signal of the label compound,
wherein the complex is a compound of formula I:

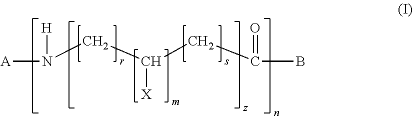

wherein A represents the label compound and B represents the analyte-specific binding agent or vice versa,
X is OH or (CHOH)ₜ—CH₂OH with t≥1, preferably t=1, 3, 5 or 7,
m is an integer from 1 to 8, preferably a ≥2, in particular 2 to 8,
n is an integer from 2 to 20, preferably 5 to 20,
r is an integer and ≥0, preferably 0, wherein r≥1 in case X=OH,
s is an integer and ≥0, preferably 0, and
z is an integer and ≥1.

The inventors surprisingly found that subject matters of the present invention, in particular the method according to the first aspect of the invention, show a complex comprising in particular a peptide-based polyol linkers, with extremely good control on structure and polydispersity. Using a single molecular weight and pure linker decreases complexity of product purification, characterization and improves reproducibility of manufacturing. In particular, the solid-phase peptide chemistry can be utilized to give a complex of the present invention.

The method as referred to in accordance with the present invention includes a method which essentially consists of the aforementioned steps or a method which includes further steps. Moreover, the method of the present invention, preferably, is an ex vivo and more preferably an in vitro method. Moreover, it may comprise steps in addition to those explicitly mentioned above. For example, further steps may relate to the detecting of further analytes of interests and/or to sample pre-treatments, enrichment steps or evaluation of the results obtained by the method. The method may be carried out manually or assisted by automation. Preferably, step (a), (b), (c) and/or (d) may in total or in part be assisted by automation, e.g., by a suitable robotic and sensory equipment.

According to step b), a complex is provided. The complex is a compound of formula I. The complex comprises a linker. The linker covalently binds a label compound and an analyte-specific binding agent. The label compound is capable of producing a detectable signal. Preferably, the detectable label is a chemiluminescence based signal.

According to step c), the sample of step a) with the complex of step b) is coupled.

According to step d), the analyte of interest is detected by using the detectable signal of the label compound.

In embodiments of the first aspect of the invention, X is OH. In this case a complex comprising a linear linker having polyol-units can be formed. Alternatively, X is (CHOH)t-CH2OH with t≥1. Preferably t=1, 3, 5 or 7, e.g. 3 or 5. In this case a complex comprising a branched linker having polyol-units can be formed.

In embodiments of the first aspect of the invention, m is an integer. m is selected from the range of 1 to 8. Preferably, m is more than or equal to 2, e.g. 2 or 3 or 4 or 5 or 6 or 7 or 8.

In embodiments of the first aspect of the invention, n is an integer. n is selected from the range of 1 to 20. Preferably, m is more than or equal to 2, e.g. 2 or 3 or 4 or 5 or 6 or 7 or 8 or 9 or 10 or 11 or 12 or 13 or 14 or 15 or 16 or 17 or 18 or 19 or 20.

In embodiments of the first aspect of the invention, r is an integer, r is more than or equal to 0. Preferably, r is 0, e.g. in case X=(CHOH)ₜ—CH₂OH with t≥1. r is more than or equal to 1 in case X=OH.

In embodiments of the first aspect of the invention, s is an integer. s is more than or equal to 0. Preferably, s is 0.

In embodiments of the first aspect of the invention, z is an integer, z is more than or equal to 1, Preferably, z is 5 to 10.

In embodiments of the first aspect of the invention, the complex is a compound of formula II:

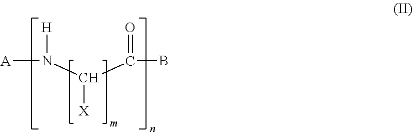

wherein each of A, B, m and n has the same meaning as mentioned above with respect to formula I,
wherein X is (CHOH)ₜ—CH₂OH with t≥1, preferably t=1, 3, 5 or 7.

In embodiments of the first aspect of the invention, the complex is a compound of formula III:

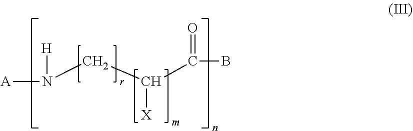

wherein each of A, B, m and n has the same meaning as mentioned above with respect to formula I,
wherein X is OH and r≥1, preferably r=1.

In embodiments of the first aspect of the invention, step b) comprises a peptide-based synthesis, preferably a solid phase peptide synthesis (SPPS).

In embodiments of the first aspect of the invention, X is OH and/or m is 2 or 4 or 6.

In embodiments of the first aspect of the invention, X is (CHOH)ₜ—CH₂OH with t=1, 3, 5 or 7.

In embodiments of the first aspect of the invention, the label compound is selected from the group consisting of an enzyme, a fluorescent dye, a luminescent dye, a metal chelate complex, and a moiety containing a radioisotope.

In embodiments of the first aspect of the invention, label compound is capable of being induced to luminesce when electrochemically oxidized or reduced.

In embodiments of the first aspect of the invention, the label compound comprises a metal ion, which is Ru²⁺ or Ir³⁺.

Preferably, the label compound is selected from the following group: Ru or Ir.

In embodiments of the first aspect of the invention, the label compound is covalently bonded to the linker via a first conjugation method, wherein the first conjugation method is selected from the following group: click chemistry, amide, ester, imide, carbonate, carbamate, squarate, thiazole, thiazolidine, hydrazone, oxime, dihydropyridazine, thiol-maleimide, cycloaddition, tetrazine ligation, photoclick, Staudinger ligation, Diels-Alder, cross-coupling, Pictet-Spengler, quadricylcane.

In embodiments of the first aspect of the invention, the analyte-specific binding agent is selected from the group consisting of an antibody, an analyte-specific fragment and/or derivative of an antibody, an aptamer, a spiegelmer, a darpin, a lectin, an ankyrin repeat containing protein, and a Kunitz type domain containing protein.

In embodiments of the first aspect of the invention, the analyte-specific binding agent is covalently bonded to the linker via a second conjugation method, wherein the second conjugation method is selected from the following group: click chemistry, amide, ester, imide, carbonate, carbamate, squarate, thiazole, thiazolidine, hydrazone, oxime, dihydropyridazine, thiol-maleimide, cycloaddition, tetrazine ligation, photoclick, Staudinger ligation, Diels-Alder, cross-coupling, Pictet-Spengler, quadricylcane.

Preferably, the analyte-specific binding agent is selected from the following group: antibody, Fab.

In embodiments of the first aspect of the invention, A of formula I, II or III represents the label compound and B of formula I, II or III represents the analyte-specific binding agent.

In embodiments of the first aspect of the invention, B of formula I, II or III represents the label compound and A of formula I, II or III represents the analyte-specific binding agent.

In embodiments of the first aspect of the invention, prior, during or after step (c) the analyte is immobilized on a solid phase.

In embodiments of the first aspect of the invention, the sample is selected from the group consisting of sputum, saliva, liquor, urine, whole blood, hemolyzed whole blood, serum and plasma.

In embodiments of the first aspect of the invention, the complex of step (b) is provided in dissolved form, and step (c) is performed in a liquid aqueous buffer.

In embodiments of the first aspect of the invention, the liquid aqueous buffer is selected from phosphate, tris buffer, citrate, cacodylate, barbital, glycine, HEPES, MES, PIPES, MOPS, bis-tris methane, ADA, bis-tris propane, ACES, MOPSO, BES, AMPB, TES, DIPSO, MOBS, acetamidoglycine, TAPSO, TEA, POPSO, HEPPSO, EPS, HEPPS, tricine, gycinamide, Gly-Gly, HEPBS, bicine, TAPS and mixtures thereof.

In embodiments of the first aspect of the invention, the liquid aqueous buffer is selected from phosphate, tris(hydroxymethyl)aminomethane (TRIS, preferably with a pH 6.0-7.4) and mixtures thereof.

In embodiments of the first aspect of the invention, the complex is a compound of formula IV-1 or IV-2:

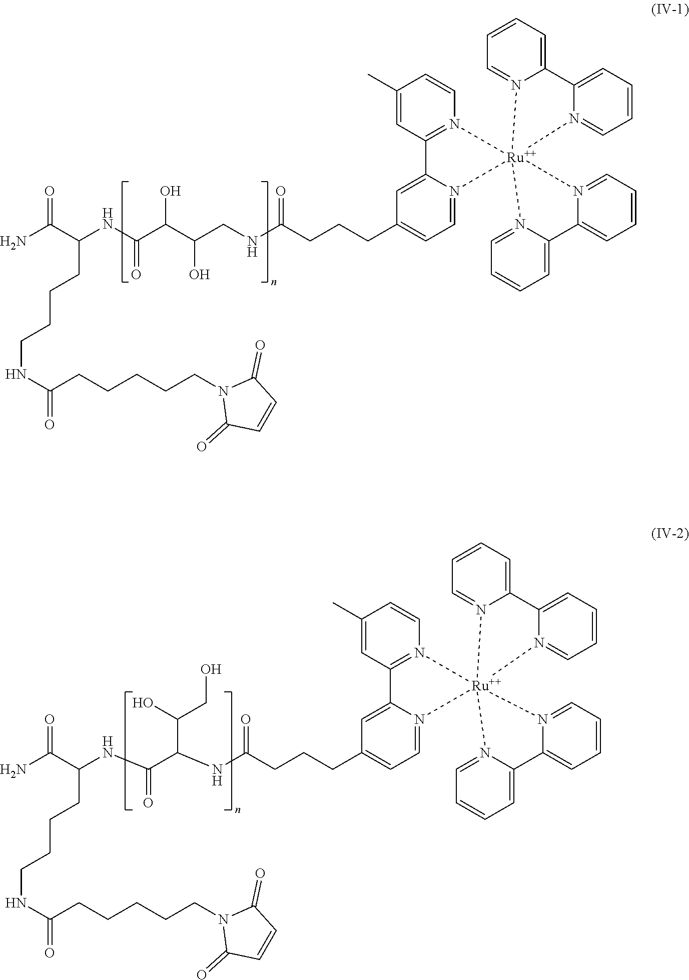

wherein n is more than 1, preferably 1≤n≤15, e.g. n=10. Preferably, the complex is a compound of the following formula:

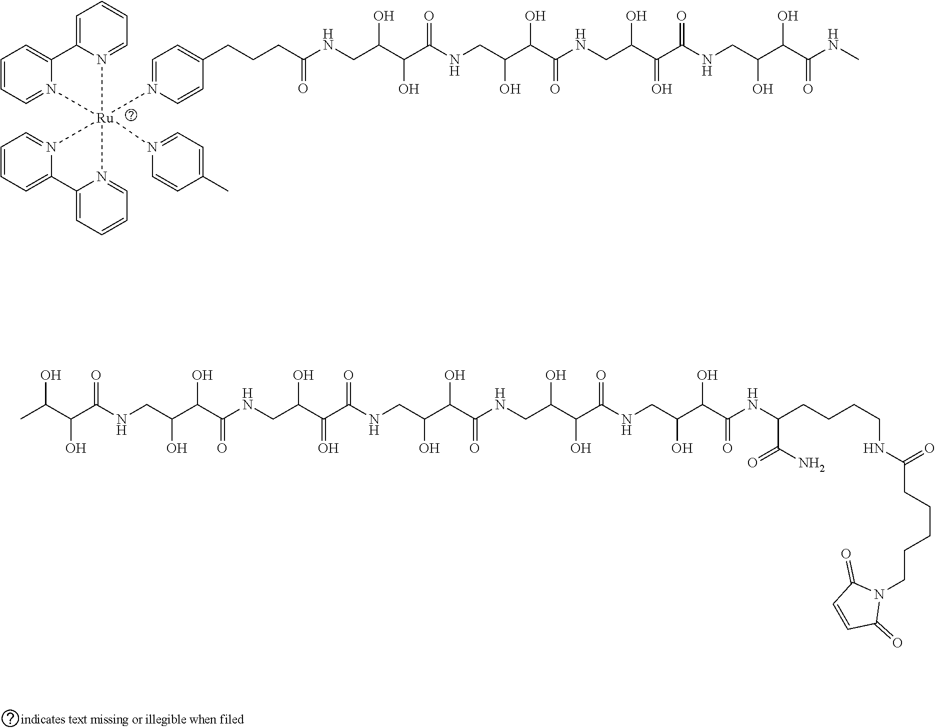

This compound is here abbreviated BPRu-(MF77)₁₀K(MH)amide.

In embodiments of the first aspect of the invention, the complex is a compound of formula V or VI:

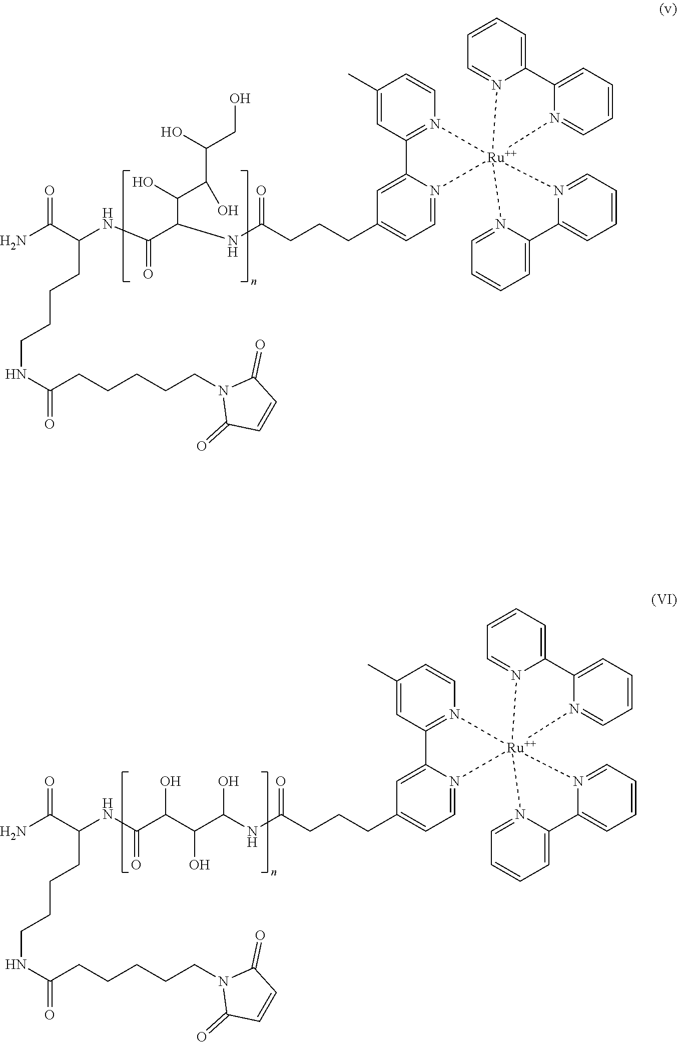

wherein n of Formula V or VI is independently of each other more than 1, preferably 1≤n≤15, e.g. n=10.

Preferably, the complex is a compound of the following formula:

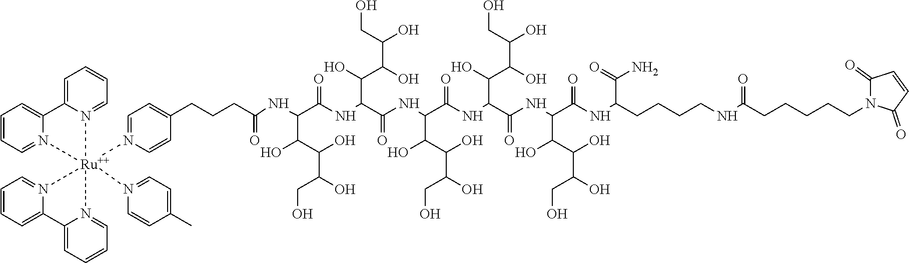

This compound is here abbreviated BPRu-(MF74)₅K(MH)amide.

Preferably, the complex is a compound of the following formula:

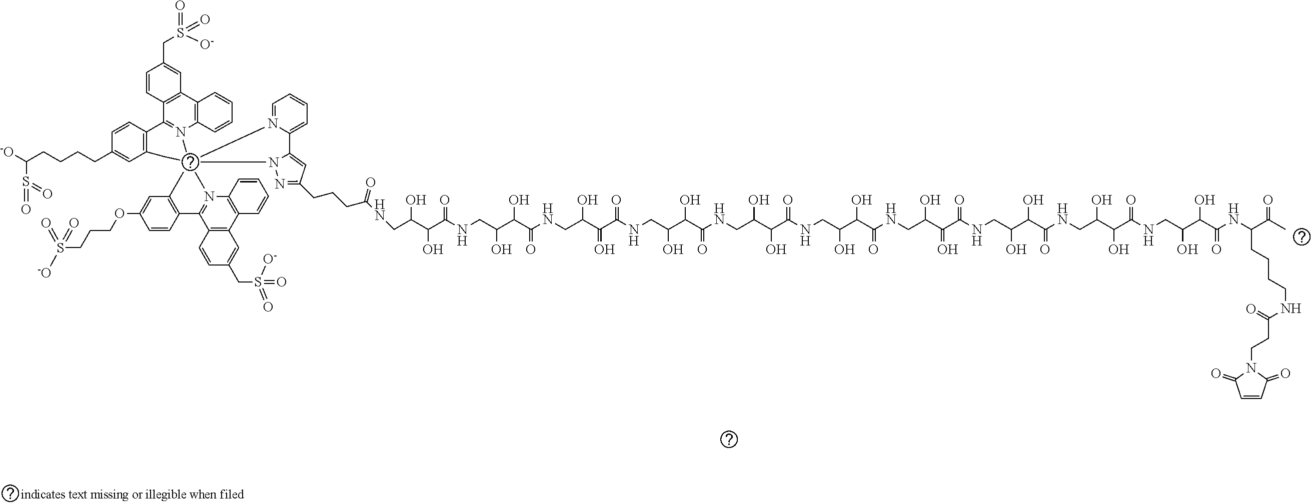

Preferably, the complex is a compound of the following formula:

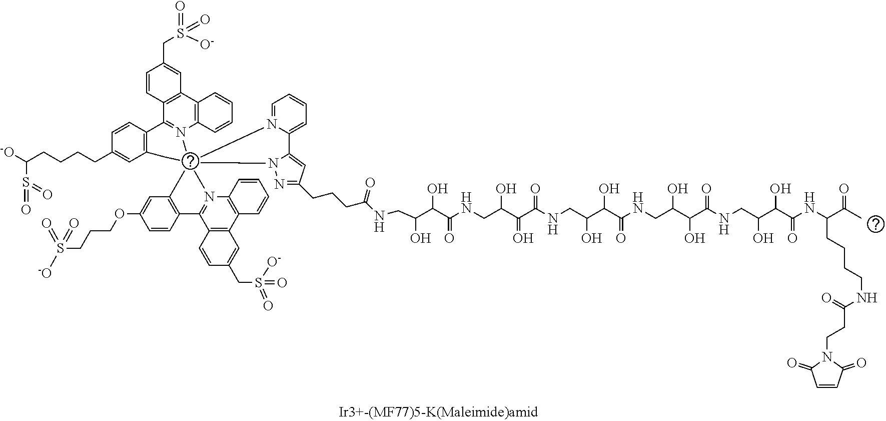

In embodiments, the label compound does not include or is free of a folic acid or derivatives thereof.

In embodiments, the label compound does not include or is free of a folate receptor binding ligand.

In embodiments, the analyte specific binding agent does not include or is free of a cysteine.

In embodiments, the method is free of drug delivery.

In embodiments, the method is a diagnostic method, preferably an in-vitro diagnostic method.

In embodiments, n>2.

In embodiments, n>4.

In embodiments, the label compound is free of a drug compound, e.g. desacetyl vinblastine hydrazide or derivatives thereof.

In embodiments, X=(CHOH)ₜ—CH₂OH with t≥1, preferably t=1, 3, 5 or 7.

In a second aspect, the present invention relates to the use of the method according to first aspect of the present invention for detecting the analyte of interest in the sample.

All embodiments mentioned for the first aspect of the invention apply for the second aspect of the invention and vice versa.

In a third aspect, the present invention relates to a kit for performing detection of an analyte of interest in a sample, the kit comprising in separate containers
a) a solid phase capable of immobilizing the analyte;
b) a compound of formula I:

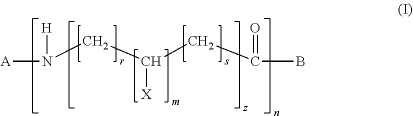

wherein A represents the label compound and B represents the analyte-specific binding agent or vice versa,
X is OH or (CHOH)ₜ—CH₂OH with t≥1, preferably t=1, 3, 5 or 7,
m is an integer from 1 to 8, preferably ≥2, in particular 2 to 8,
n is an integer from 2 to 20, preferably 5 to 20,
r is an integer and ≥0, preferably 0, wherein r≥1 in case X=OH.
s is an integer and ≥0, preferably 0, and
z is an integer and ≥1.

All embodiments mentioned for the first aspect of the invention and/or second aspect of the invention apply for the third aspect of the invention and vice versa.

In embodiments of the third aspect of the invention, the complex is a compound of formula II:

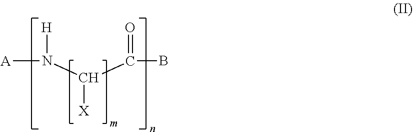

wherein each of A, B, X, m and n has the same meaning as mentioned in aspect 21.
wherein X is (CHOH)ₜ—CH₂OH with t≥1, preferably t=1, 3, 5 or 7.

In embodiments of the third aspect of the invention, the complex is a compound of formula III:

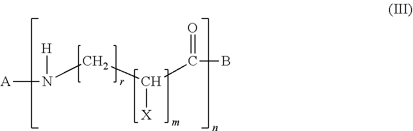

wherein each of A, B, m and n has the same meaning as mentioned in aspect 21,
wherein X is OH and r≥1, preferably r=1.

In embodiments of the third aspect of the invention, X is OH and in is 2 or 4 or 6.

In embodiments of the third aspect of the invention, X is (CHOH)ₜ—CH₂OH with t=1, 3, 5 or 7.

In embodiments of the third aspect of the invention, the complex is embodied in dissolved form.

In embodiments of the third aspect of the invention, the at least one container or containers is made of glass or plastic.

In a fourth aspect, the present invention relates to the use of the kit according to third aspect of the present invention for detecting the analyte of interest in the sample.

All embodiments mentioned for the first aspect of the invention and/or second aspect of the invention and/or third aspect of the invention apply for the fourth aspect of the invention and vice versa.

In a fifth aspect, the present invention relates to A complex of formula I,

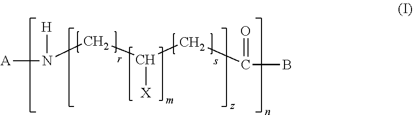

wherein A represents the label compound and B represents the analyte-specific binding agent or vice versa,
X is OH or (CHOH)ₜ—CH₂OH with t≥1, preferably t=1, 3, 5 or 7,
m is an integer from 1 to 8, preferably ≥2, in particular 2 to 8,
n is an integer from 2 to 20, preferably 5 to 20,
r is an integer and ≥0, preferably 0, wherein r≥1 in case X=OH,
s is an integer and ≥0, preferably 0, and
z is an integer and ≥1,
preferably wherein the compound is suitable to detect an analyte of interest in a sample.

All embodiments mentioned for the first aspect of the invention and/or second aspect of the invention and/or third aspect of the invention and/or fourth aspect of the invention apply for the fifth aspect of the invention and vice versa.

In embodiments of the fifth aspect of the invention, the complex is a compound of formula II:

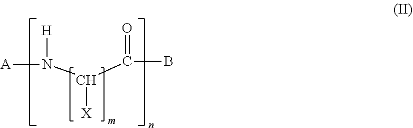

wherein each of A, B, X, m and n has the same meaning as mentioned in aspect 28,
wherein X is (CHOH)ₜ—CH₂OH with t≥1, preferably t=1, 3, 5 or 7.

In embodiments of the fifth aspect of the invention, the complex is a compound of formula III:

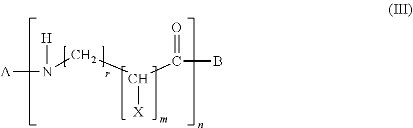

wherein each of A, B, m and n has the same meaning as mentioned in aspect 28,
wherein X is OH and r≥1, preferably r=1.

In embodiments of the fifth aspect of the invention. X is OH and in is 2 or 4 or 6.

In embodiments of the fifth aspect of the invention, X is (CHOH)ₜ—CH₂OH with t=1, 3, 5 or 7.

In embodiments of the fifth aspect of the invention, A or B is selected from the group consisting of a peptide, a polypeptide, and a protein.

In embodiments of the fifth aspect of the invention, A comprises the analyte-specific binding agent and B comprises the label compound, or B comprises the analyte-specific binding agent and A comprises the label compound.

In embodiments of the fifth aspect of the invention, the analyte-specific binding agent is selected from the group consisting of an antibody, an analyte-specific fragment and/or derivative of an antibody, an aptamer, a spiegelmer, a darpin, a lectin, an ankyrin repeat containing protein, and a Kunitz type domain containing protein, and the label compound is selected from the group consisting of enzyme, a fluorescent dye, a luminescent dye, a metal chelate complex, and a moiety containing a radioisotope.

In a sixth aspect, the present invention relates to a method to synthesize a complex of the fifth aspect of the present invention comprising the steps of
a) Providing a monomer or derivatives thereof, wherein the monomer is an amino acid comprising an amino group, a carboxy group and at least one hydroxyl group, wherein the amino group or the carboxy group is protected by a first protecting group, and the at least one or each hydroxyl group is protected by a second protecting group,
b) Using the monomer in a process of solid phase peptide synthesis, cleaving the first and second protection group and forming a complex of formula III,

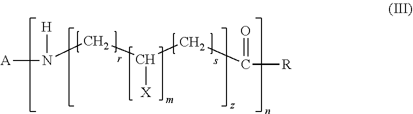

wherein A represents the label compound and R represents a second spacer or vice versa, wherein R is capable of covalently bonding to an analyte-specific binding agent or is covalently bonded to an analyte-specific binding agent,
X is OH or (CHOH)ₜ—CH₂OH with t≥1, preferably t=1, 3, 5 or 7.
m is an integer from 1 to 8, preferably 2, in particular 2 to 8,
n is an integer from 2 to 20, preferably 5 to 20,
r is an integer and ≥0, preferably 0, wherein r≥1 in case X=OH,
s is an integer and ≥0, preferably 0, and
z is an integer and ≥1.

All embodiments mentioned for the first aspect of the invention and/or second aspect of the invention and/or third aspect of the invention and/or fourth aspect of the invention and/or fifth aspect of the invention apply for the sixth aspect of the invention and vice versa.

In embodiments of the sixth aspect of the invention, the first and/or second the first and/or second protecting group is selected from the group ester, ether, silylether, acetal, 9-fluorenylmethyloxycarbonyl (Fmoc), benzyloxycarbonyl (Boc), benzyloxycarbonyl (Cbz), allyloxycarbonyl (Alloc), amide and tert butyl ester.

In embodiments of the sixth aspect of the invention, a monomer or a derivative thereof is selected from the following, formulae m-1 to m-4:

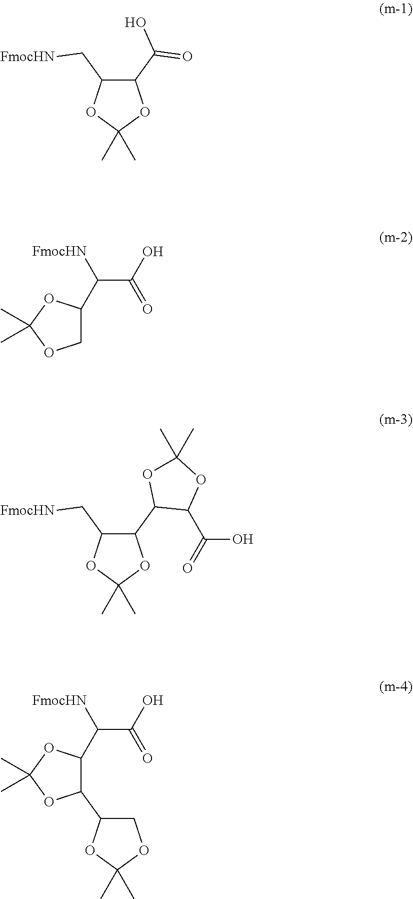

wherein FmocHN means an amine protected with a 9-fluorenylmethoxycarbonyl protecting group. The abbreviation m-1 can also here and in the content of this disclosure be named as MF77. The abbreviation m-2 can also here and in the content of this disclosure be named as (compound) 18 (see e.g. scheme 4) or FA36. The abbreviation m-3 can also here and in the content of this disclosure be named as S779. The abbreviation m-4 can also here and in the content of this disclosure be named as MF74.

In embodiments of the sixth aspect of the invention, the complex of formula III as to presented above can be synthesized in and provided using an automated solid phase peptide synthesis (SPPS) device by employing standard knowledge and processes, methods and techniques known to the art of solid phase synthesis.

In further embodiments, the present invention relates to the following aspects:
1. A method for detecting an analyte of interest in a sample comprising the steps of
   a) Providing the sample comprising the analyte of interest,
   b) Providing a complex comprising a linker, wherein the linker covalently binds a label compound and an analyte-specific binding agent, wherein the label compound is capable of producing a detectable signal, preferably a chemiluminescence based signal,
   c) Coupling the sample of step a) with the complex of step b),
   d) Detecting the analyte of interest by using the detectable signal of the label compound,
   wherein the complex is a compound of formula I:

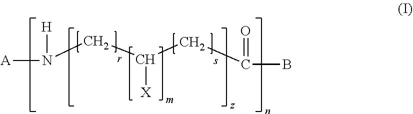

wherein A represents the label compound and B represents the analyte-specific binding agent or vice versa,
X is OH or (CHOH)ₜ—CH₂OH with t≥1, preferably t=1, 3, 5 or 7,
m is an integer from 1 to 8, preferably ≥2, in particular 2 to 8.
n is an integer from 2 to 20, preferably 5 to 20,
r is an integer and ≥0, preferably 0, wherein r≥1 in case X=OH,
s is an integer and ≥0, preferably 0, and
z is an integer and ≥1.
2. The method of aspect 1, wherein the complex is a compound of formula II:

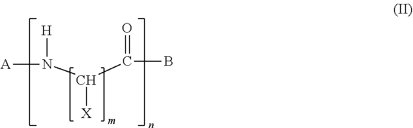

wherein each of A, B, m and n has the same meaning as mentioned in aspect 1,
wherein X is (CHOH)ₜ—CH₂OH with t≥1, preferably t=1, 3, 5 or 7.
3. The method of aspect 1, wherein the complex is a compound of formula III:

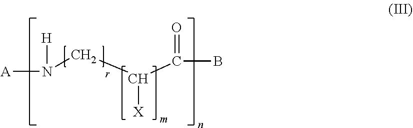

wherein each of A, B, m and n has the same meaning as mentioned in aspect 1,
wherein X is OH and r≥1, preferably r=1.
4. The method of any of the preceding aspects, wherein X is OH and/or m is 2 or 4 or 6.
5. The method of any of the preceding aspects, wherein X is (CHOH)ₜ—CH₂OH with t=1, 3, 5 or 7.
6. The method of any of the preceding aspects, wherein the label compound is selected from the group consisting of an enzyme, a fluorescent dye, a luminescent dye, a metal chelate complex, and a moiety containing a radioisotope.
7. The method of any of the preceding aspects, wherein the label compound is capable of being induced to luminesce when electrochemically oxidized or reduced.
8. The method of any of the preceding aspects, wherein the label compound comprises a metal ion, which is Ru²⁺ or Ir³⁺.
9. The method of any of the preceding aspects, wherein the label compound is covalently bonded to the linker via a first conjugation method, wherein the first conjugation method is selected from the following group: click chemistry, amide, ester, imide, carbonate, carbamate, squarate, thiazole, thiazolidine, hydrazone, oxime, dihydropyridazine, thiol-maleimide, cycloaddition, photoclick, Staudinger ligation, Diels-Alder, tetrazine ligation, cross-coupling, Pictet-Spengler, quadricylcane.
10. The method of any of the preceding aspects, wherein the analyte-specific binding agent is selected from the group consisting of an antibody, an analyte-specific fragment and/or derivative of an antibody, an aptamer, a spiegelmer, a darpin, a lectin, an ankyrin repeat containing protein, and a Kunitz type domain containing protein.
11. The method of any of the preceding aspects, wherein the analyte-specific binding agent is covalently bonded to the linker via a second conjugation method, wherein the second conjugation method is selected from the following group: click chemistry, amide, ester, imide, carbonate, carbamate, squarate, thiazole, thiazolidine, hydrazone, oxime, dihydropyridazine, thiol-maleimide, cycloaddition, tetrazine ligation, photoclick, Staudinger ligation, Diels-Alder, cross-coupling, Pictet-Spengler, quadricylcane.
12. The method of any of the preceding aspects, wherein A of formula I, II or III represents the label compound and B of formula I, II or III represents the analyte-specific binding agent.
13. The method of any of the preceding aspects, wherein B of formula I, II or III represents the label compound and A of formula I, II or III represents the analyte-specific binding agent,
14. The method of any of the preceding aspects, wherein prior, during or after step (c) the analyte is immobilized on a solid phase.
15. The method of any of the preceding aspects, wherein the sample is selected from the group consisting of sputum, saliva, liquor, urine, whole blood, hemolyzed whole blood, serum and plasma.
16. The method of any of the preceding aspects, wherein the complex of step (b) is provided in dissolved form, and step (c) is performed in a liquid aqueous buffer.
17. The method of any of the preceding aspects, wherein the liquid aqueous buffer is selected from phosphate, tris buffer, citrate, cacodylate, barbital, glycine, HEPES, MES, PIPES. MOPS, bis-tris methane, ADA, bis-tris propane, ACES, MOPSO, BES, AMPB, TES, DIPSO, MOBS, acetamidoglycine. TAPSO, TEA, POPSO, HEPPSO, EPS, HEPPS, tricine, gycinamide, Gly-Gly, HEPBS, bicine, TAPS and mixtures thereof.
18. The method of any of the preceding aspects, wherein the complex is a compound of formula IV-1:

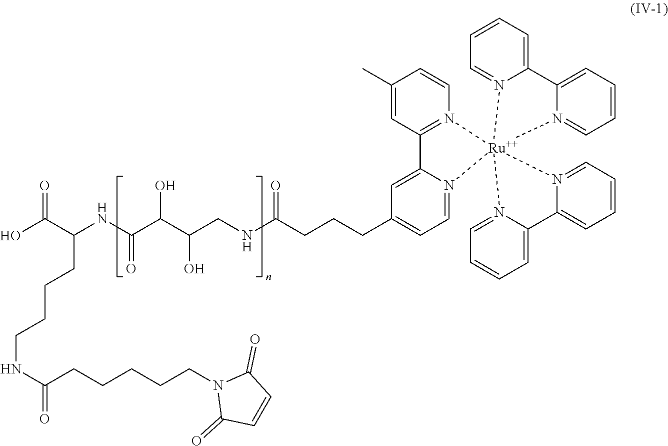

wherein n is more than 1, preferably 1≤n≤15, e.g. n=10.
19. The method of any of the preceding aspects, wherein the complex is a compound of formula V or VI:

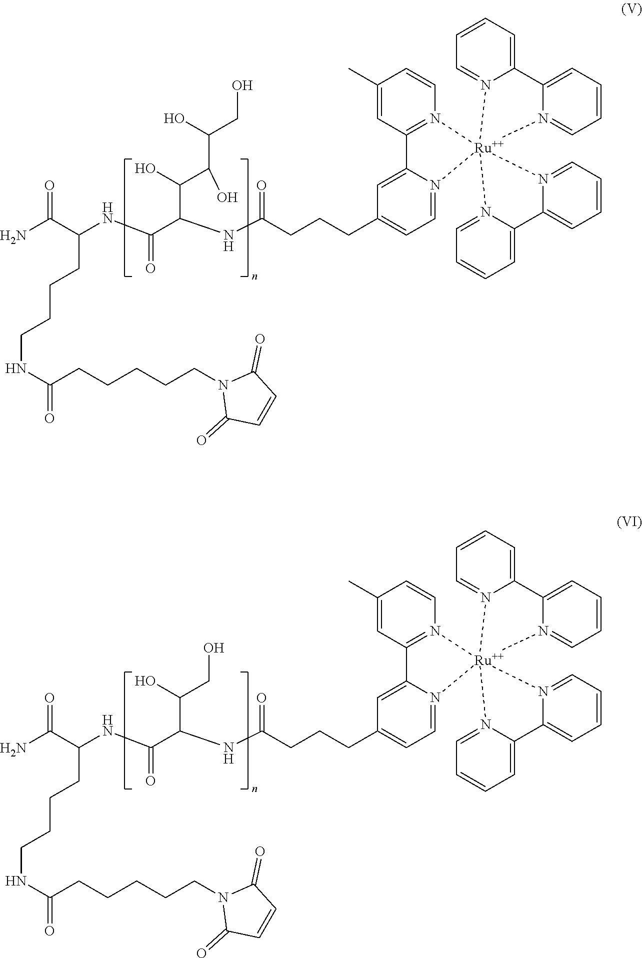

wherein n of formula V or VI is independently of each other more than 1, preferably 1≤n≤15, e.g. n=10.
20. The method of any of the preceding aspects, wherein step b) comprises a peptide-based synthesis, preferably a solid phase peptide synthesis (SPPS).
21. Use of the method according to any of the proceeding aspects 1 to 20 for detecting the analyte of interest in the sample.
22. A kit for performing detection of an analyte of interest in a sample, the kit comprising in separate containers
a) a solid phase capable of immobilizing the analyte;
b) a compound of formula I:

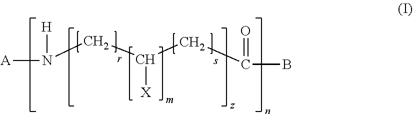

wherein A represents the label compound and B represents the analyte-specific binding agent or vice versa,
X is OH or (CHOH)ₜ—CH₂OH with t≥1, preferably t=1, 3, 5 or 7,
m is an integer from 1 to 8, preferably ≥2, in particular 2 to 8,
n is an integer from 2 to 20, preferably 5 to 20,
r is an integer and ≥0, preferably 0, wherein r≥1 in case X=OH,
s is an integer and ≥0, preferably 0, and
z is an integer and ≥1.
23. The kit of aspect 22, wherein the complex is a compound of formula II:

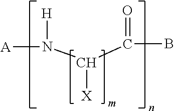

wherein each of A, B, X, m and n has the same meaning as mentioned in aspect 22.
wherein X is (CHOH)ₜ—CH₂OH with t≥1, preferably t=1, 3, 5 or 7.
24. The kit of aspect 22, wherein the complex is a compound of formula III:

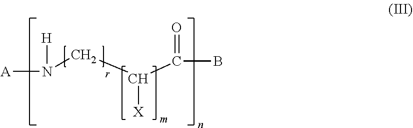

wherein each of A, B, m and n has the same meaning as mentioned in aspect 22,
wherein X is OH and r≥1, preferably r=1.
25. The kit of any of the aspect 22 to 24, wherein X is OH and m is 2 or 4 or 6.
26. The kit of any of the aspect 22 to 25, wherein X is (CHOH)ₜ—CH₂OH with t=1, 3, 5 or 7.
27. The kit of any of the aspect 22 to 26, wherein the complex is embodied in dissolved form.
28. Use of the kit according to any of the proceeding claims 22 to 27 for detecting the analyte of interest in the sample.
29. A complex of formula I,

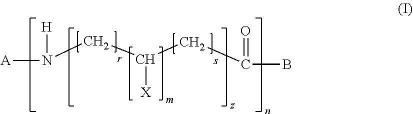

wherein A represents the label compound and B represents the analyte-specific binding agent or vice versa,
X is OH or (CHOH)ₜ—CH₂OH with t≥1, preferably t=1, 3, 5 or 7,
m is an integer from 1 to 8, preferably ≥2, in particular 2 to 8,
n is an integer from 2 to 20, preferably 5 to 20,
r is an integer and ≥0, preferably 0, wherein r≥1 in case X=OH
s is an integer and ≥0, preferably 0, and
2 is an integer and ≥1.
preferably wherein the compound is suitable to detect an analyte of interest in a sample.
30. The complex of aspect 29, wherein the complex is a compound of formula II:

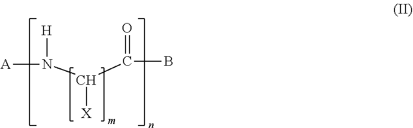

wherein each of A, B, X, m and n has the same meaning as mentioned in aspect 29,
wherein X is (CHOH)ₜ—CH₂OH with t≥1, preferably t=1, 3, 5 or 7.
31. The complex of aspect 29, wherein the complex is a compound of formula III:

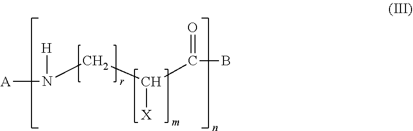

wherein each of A, B, m and n has the same meaning as mentioned in aspect 29,
wherein X is OH and r≥1, preferably r=1.
32. The complex of any of aspects 29 to 31, wherein X is OH and m is 2 or 4 or 6.
33. The complex of any of aspects 29 to 32, wherein X is (CHOH)ₜ—CH₂OH with t=1, 3, 5 or 7.
34. The complex of any of aspects 29 to 33, wherein A or B is selected from the group consisting of a peptide, a polypeptide, and a protein.
35. The complex of any of aspects 29 to 34, wherein A comprises the analyte-specific binding agent and B comprises the label compound, or B comprises the analyte-specific binding agent and A comprises the label compound.
36. The complex of any of aspects 29 to 35, wherein the analyte-specific binding agent is selected from the group consisting of an antibody, an analyte-specific fragment and/or derivative of an antibody, an aptamer, a spiegelmer, a darpin, a lectin, an ankyrin repeat containing protein, and a Kunitz type domain containing protein, and the label compound is selected from the group consisting of enzyme, a fluorescent dye, a luminescent dye, a metal chelate complex, and a moiety containing a radioisotope.
37. A method to synthesize a complex ofany of aspects 29 to 36 comprising the steps of
a) Providing a monomer or derivatives thereof, wherein the monomer is an amino acid comprising an amino group, a carboxy group and at least one hydroxyl group, wherein the amino group or the carboxy group is protected by a first protecting group, and the at least one or each hydroxyl group is protected by a second protecting group,
b) Using the monomer in a process of solid phase peptide synthesis, cleaving the first and second protection group and forming a complex of formula III,

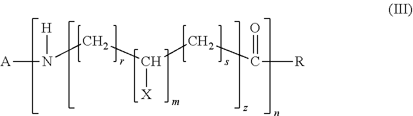

wherein A represents the label compound and R represents a second spacer or vice versa, wherein R is capable of covalently bonding to an analyte-specific binding agent or is covalently bonded to an analyte-specific binding agent,
X is OH or (CHOH)ₜ—CH₂OH with t≥1, preferably t=1, 3, 5 or 7,
m is an integer from 1 to 8, preferably ≥2, in particular 2 to 8,
n is an integer from 2 to 20, preferably 5 to 20,
r is an integer and ≥0, preferably 0, wherein r≥1 in case X=OH,
s is an integer and ≥0, preferably 0, and
z is an integer and ≥1.
38. The method of aspect 37, wherein the first and/or second protecting group is selected from the group Fmoc, tBu (tert-buthyl) and ether, ester and/or acetal.
39. The method ofany of aspects 37 to 38, wherein a monomer or a derivative thereof is selected from the following formulae m-1 to m-4:

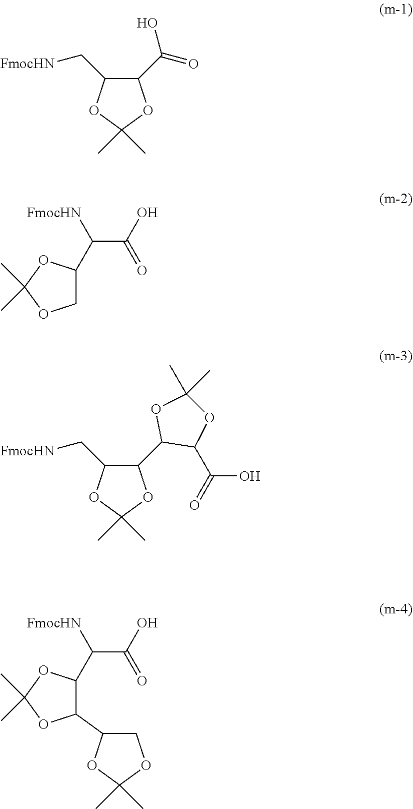

wherein FmocHN means an amine protected with a 9-fluorenylmethoxycarbonyl protecting group.

### EXAMPLES

The following examples are provided to illustrate, but not to limit the presently claimed invention.

### Example 1

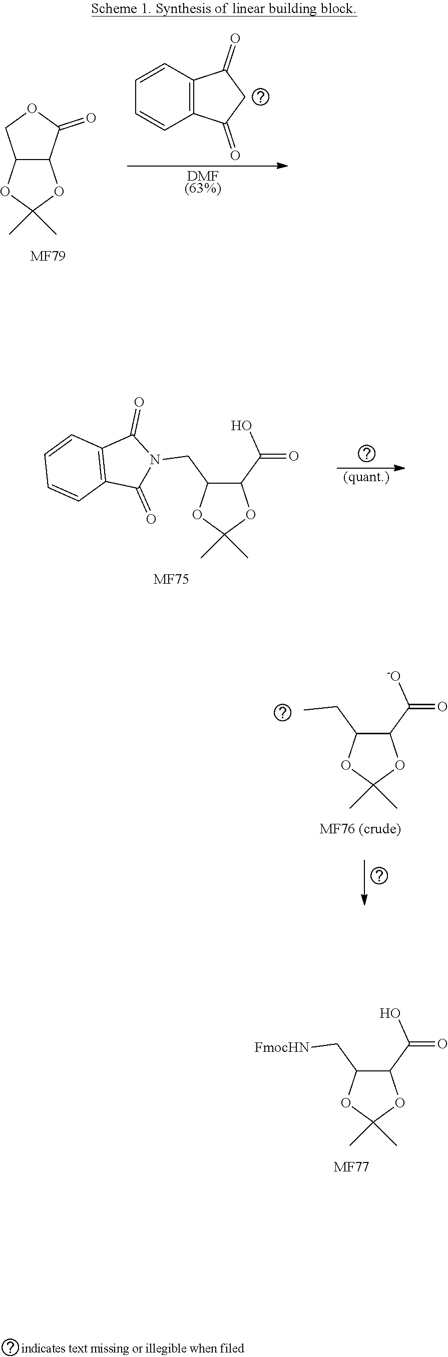

Amino acid (MF76) was obtained from commercially available (-)-2,3-O-isopropylidene-D-erythronolactone (MF79) by the method of Kamiya et al (Kamiya, T.; Saito. Y.; Hashimoto, M.; Seki, H. Tetrahedron 1972, 28, 899). Subsequent Fmoc protection with Fmoc-OSu using standard MeCN—aqueous Na₂CO₃ conditions furnished building block MF77 in 81% yield, MF: C22H23NO6. MW: 397.15. Physical State: white solid. ¹H NMR (CDCl₃, 400 MHz): δ=7.77 (d, J=7.5 Hz, 2H), 7.60 (d, J=7.5 Hz, 2H), 7.37-7.44 (m, 2H), 7.30-7.36 (m, 2H), 5.22-5.37 (m, 1H), 4.65 (d, J=7.3 Hz, 1H), 4.36-4.58 (m, 3H), 4.17-4.31 (m, 1H), 3.45-3.53 (m, 2H), 1.61 (s, 3H), 1.42 (s, 3H) ppm. HPLC-MS (TEAAc(pH 7.0)—MeCN, gradient in 7 min from 5% to 100% of McCN) m/z 396.2 ([M-H]⁻); retention time 4.8 min.

### Example 2

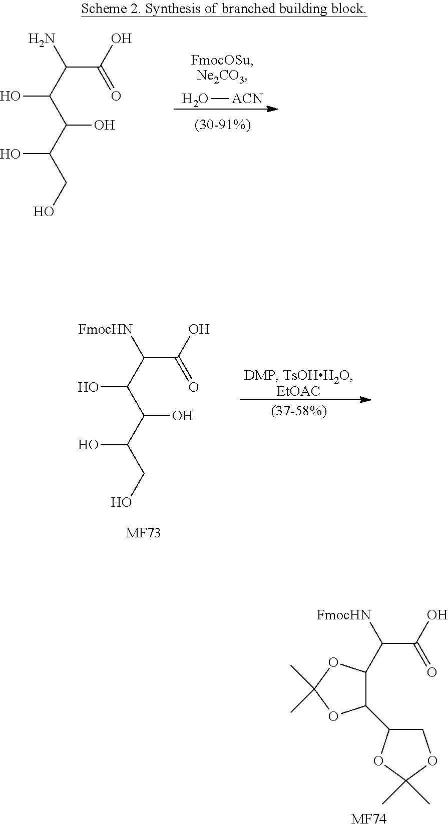

Fmoc-D-glucosaminic acid (MF73). Fmoc-OSu (3.28 g, 9.74 mmol) in MeCN (50 mL) was added to a stirred solution of D-glucosaminic acid (2.0 g, 10.25 mmol) and potassium carbonate (2.17 g, 20.50 mmol) in water (50 mL) at 0° C. The mixture briefly turned clear before white turbidity formed. The reaction was stirred at 0° C. for 0.5 h, then at RT for 1 h. The mixture was adjusted to pH 8 by the addition of HCl (1.0 M), and then concentrated in vacuo. The residue was taken up in water (−200 mL) and extracted with EtOAc (3×100 mL). The aqueous layer was adjusted to pH 2.0 by the addition of HCl (1.0 M) and extracted with EtOAc (5×150 mL). The combined organic layers were washed with brine, dried (Na₂SO₄), filtered, and concentrated to obtain 4.0 g of the desired product (yield 94%). MF: C21H23NOS. MW: 417.14. Physical State: white solid. HPLC-MS (TEAAc (pH 7.0)—MeCN, gradient in 7 min from 5% to 100% of MeCN) m/z 416.2 ([M-H]−); retention time 4.1 min.

Fmoc-isopropylidene-D-glucosaminic acid (MF74). To a stirred suspension of Fmoc-D-glucosaminic acid MF73 (1.22 g, 2.92 mmol) in EtOAc (20 mL) was added 2,2-dimethoxypropane (20 mL) and p-toluenesulfonic acid (01 g, 0.53 mmol). After 1 h the solution was diluted with EtOAc(200 mL) and washed with brine(3×25 mL). The organic layer was separated, dried (Na₂SO₄), and concentrated. The residue was converted to Na-salt, which was purified by flash chromatography (C-18, H₂O-MeCN) to give 840 ng of the desired product (yield 58%). MF: C27H31 NO8. MW: 497.20. Physical State: white solid. ¹H NMR (CDCl₃, 400 MHz): δ=7.77 (d, J=7.4 Hz, 2H), 7.62 (dd, J=8.2, 8.0 Hz, 2H), 7.41 (dd, J=7.5, 7.4 Hz, 2H), 7.32 (dd, J=7.4, 7.3 Hz, 2H), 5.59 (d, J=10.0 Hz, 1H), 4.79 (d, J=9.9 Hz, 1H), 4.46-4.55 (m, 2H), 4.41 (dd, J=10.5, 7.5 Hz, 1H), 4.26 (dd, J=7.0, 6.9 Hz, 1H), 4.09-4.23 (m, 2H), 4.01 (dd, J=8.0, 3.5 Hz, 1H), 3.72 (t, J=7.9 Hz, 1H), 1.44 (s, 3H), 1.43 (s, 3H), 1.40 (s, 3H), 1.36 (s, 3H) ppm. HPLC-MS (TEAAc(pH 7.0)—MeCN, gradient in 7 min from 5% to 100% of MeCN) m/z 496.3 ([M-H]−); retention time 5.3 min.

### Example 3

Example 3 shows the synthesis of extended linear building block (Scheme 3).

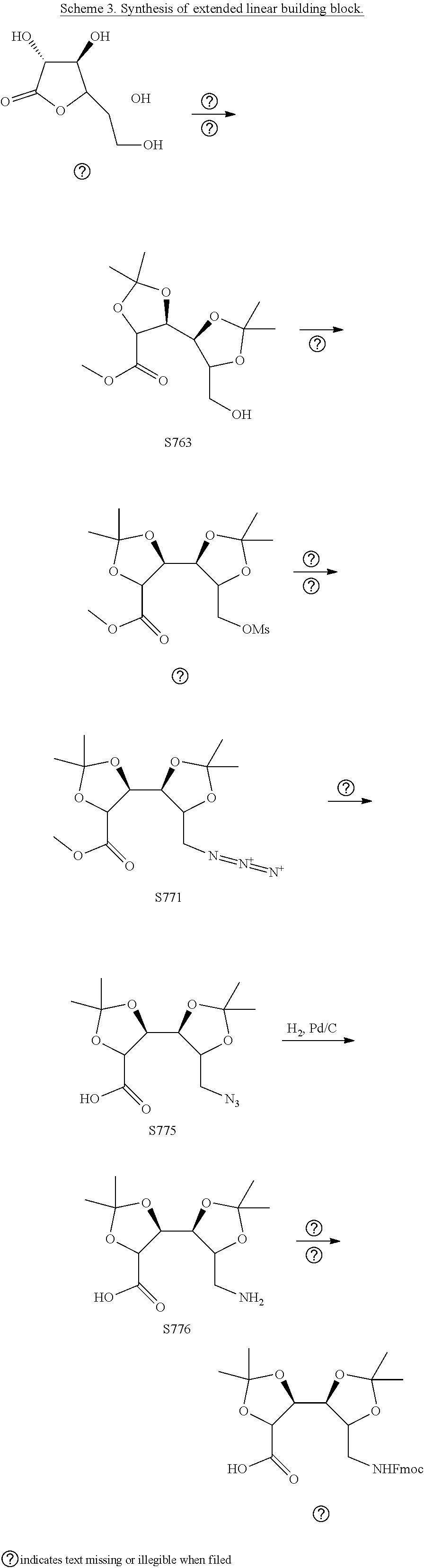

### Synthesis of Compound S763

D-Galactono-1,4-lactone (2.94 g, 16.5 mmol) was suspended in 2,2-dimethoxypropane (60 mL) and acetone (4.5 mL) and after that p-toluensulfonic acid monohydrate (1.59 g, 8.35 mmol) was added and the reaction mixture was stirred at 40° C. for 7 h. The reaction was then quenched by adding Na2CO3, the suspension was filtered over a pad of celite and the solvent was removed at reduced pressure.

The residue was then dissolved in dichloromethane and the organic layer was washed twice with water, dried over anhydrous Na₂SO₄, and concentrated at reduced pressure. Finally the residue was purified by flash column chromatography (SiO₂, n-hexane/EtOAc 1:1) obtaining 2.59 g of the desired product (54%) ¹H NMR (400 MHz, CHLOROFORM-d) δ ppm 1.36-1.43 (m, 9H) 1.46 (s, 3H) 3.67-3.75 (m, 1H) 3.76-3.80 (m, 3H) 3.80-3.87 (m, 1H) 3.90-3.96 (m, 1H) 4.05-4.12 (m, 1H) 4.33-4.39 (m, 1H) 4.53-4.57 (m, 1H) 7.21-7.37 (m, 2H)

### Synthesis of Compound 8769

Compound S763 (2.59 g, 8.92 mmol) was dissolved in 35 ml, dry Pyridine and after cooling the solution to 0° C. with an ice bath DMAP(115 mg, 0.94 mmol) was added. Finally, mesyl chloride (828 μL, 10.71 mmol) was added and the mixture was stirred 1 h at 0° C. and an additional h at rt. The solvent was evaporated at reduced pressure and the residue was dissolved in dichlormethane. The organic phase was then washed with water, NaCl saturated solution and then dried over anhydrous Na2SO4. The solvent was evaporated at reduced pressure and the residue was purified by flash column chromatography (SiO2, n-hexane:EtOAc 7:3) obtaining 2.93 g of the desired product (89%). ¹H NMR (400 MHz, CHLOROFORM-d) δ ppm 1.41-1.48 (m, 12H) 3.09 (s, 3H) 3.81 (s, 3H) 3.94 (t, J=7.53 Hz, 1H) 4.24-4.30 (m, 2H) 4.30-4.42 (m, 2H) 4.50 (dd, J=11.10, 2.70 Hz, 1H) 4.56 (d, J=5.40 Hz, 1H)

### Synthesis of compound S771

Compound S769 (2.93 g, 7.961 mmol) was dissolved in dry DMF (27 mL) and then Na N₃ (569 mg, 8.757 mmol) was added and the mixture was stirred at 85° C. for 5 h. The solvent was evaporated at reduced pressure and the residue distributed in a mixture of H₂O and EtOAc. After separating the two phases, the aqueous layer was extracted twice with EtOAc and the combined organic layer was washed with NaCl saturated solution, dried over anhydrous Na2SO4, and concentrated to dryness. The residue was finally purified by flash column chromatography (SiO2, n-hexane:EtOac 9:1) giving 1.74 of product (69%). ¹H NMR (400 MHz, CHLOROFORM-d) δ ppm 1.35-1.53 (m, 12H) 3.32 (dd, J=13.18, 5.02 Hz, 1H) 3.64 (dd, J=13.18, 3.26 Hz, 1H) 3.79-3.80 (m, 3H) 3.94 (t, J=7.50 Hz, 1H) 4.12-4.20 (m, 1H) 4.34 (dd, J=7.53, 5.27 Hz, 1H) 4.55 (d, J=5.27 Hz, 1H)

### Synthesis of Compound S775

Compound S771(1.74 g, 5.518 mmol) was dissolved in a mixture 1:2 THF/H₂O (160 mL) and after that Ba(OH)₂·8 H2O (2.84 g, 16.5 mmol) was added and the mixture was stirred at rt for 1 h. DOWEX 50WX2 was added and after that the mixture was filtered. Finally the filtrate was dried in vacuo and directly used for the following step.

### Synthesis of Compound S777

Compound S775 (1.37 g, 4.54 mmol) was dissolved in a MeOH/H₂O 4:1 mixture (25 mL), and then Pd on activated carbon was added. The flask was then evacuated and set under H₂ atmosphere and the mixture was vigorously stirred for 3 h at rt constantly supplying H₂. The mixture was filtered on a pad of celite and the filtrate was concentrated at reduced pressure and dried in vacuo, obtaining 1.25 mg of product, which were directly used for the following step (100%). ¹H NMR (400 MHz, METHANOL-d₄) δ ppm 1.40-1.46 (m, 12H) 3.06 (dd, J=13.20, 8.80 Hz, 1H) 3.24 (dd, J=13.10, 2.90 Hz, 1H) 4.01 (dd, J=7.90, 4.40 Hz, 1H) 4.25 (d, J=6.60 Hz, 1H) 4.29-4.38 (m, 2H)

### Synthesis of Compound S779

Compound S777 (1.15 g, 4.17 mmol) was dissolved in a H₂O/Acetone 1:1 mixture (100 mL) and after that FmocOSu (2.28 g, 6.75 mmol) was added and the mixture was stirred overnight at rt. NaHCO₃ (350 mg, 4.18 mmol) was added and after stirring for 1 h at rt the pH was adjusted to ≈5 units using 0.1 M HCl and the mixture was extracted 3 times with EtOAc. The combined organic layer was dried on anhydrous Na₂SO₄, concentrated at reduced pressure, and the residue was purified by flash column chromatography (SiO₂, gradient elution from n-hexane:EtOAc 2:3 to EtOAc+1% acetic acid) obtaining 1.5 g of the desired product (73%). ¹H NMR (400 MHz, CHLOROFORM-d) δ ppm 1.34-1.50 (m, 12H) 3.42-3.56 (m, 2H) 3.75-3.84 (m, 1H) 4.08-4.16 (m, 1H) 4.17-4.24 (m, 1H) 4.27-4.35 (m, 1H) 4.38-4.48 (m, 2H) 4.59 (d, J=6.00 Hz, 1H) 5.15-5.25 (m, 1H) 7.30 (t, J=7.50 Hz, 2H) 7.39 (t, J=7.40 Hz, 2H) 7.58 (d, J=7.40 Hz, 2H) 7.75 (d, J=7.60 Hz, 2H)

### Example 3b

### Synthesis of Branched Diol Derivative

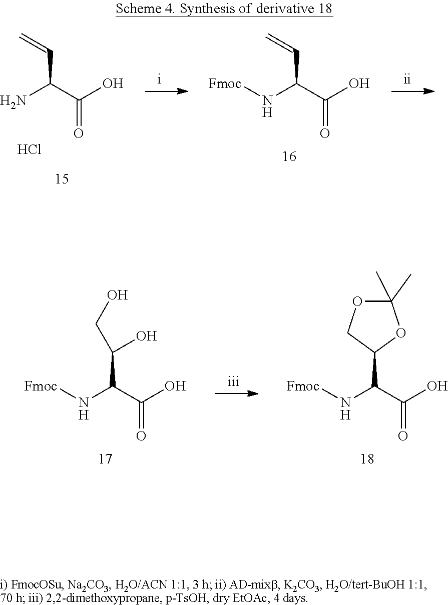

### Synthesis of Compound 16

(S)-2-aminobut-3-enoic acid hydrochloride (15, 750 mg, 5.45 mmol) was dissolved in a H₂O/ACN 1:1 mixture (50 mL) and then FmocOSu (1.84 g, 5.45 mmol) and Na₂CO₃ (1.73 g, 16.3 mmol) were added. The reaction mixture was stirred for 3 h at r.t. and, after evaporating the organic solvent at reduced pressure, the aqueous solution was acidified to pH≈1-2 using 2N HCl. The obtained mixture was then extracted 5 times with EtOAc and the organic layer was dried over anhydrous Na₂SO₄ and concentrated at reduced pressure. The raw product was purified by flash-column chromatography (RP-C18AQ, H₂O/ACN gradient elution from 2:3 to 3:2). The fractions containing the product were pooled and the organic solvent was evaporated at reduced pressure. The white precipitate formed was filtered off and dried in vacuo, obtaining 1.50 g of the Fmoc-protected derivative 16(85%); ¹H NMR (400 MHz, CHLOROFORM-d) δ ppm 4.18-4.56 (m, 3 H) 4.60-5.55 (m, 4H) 5.76-6.06 (m, 1H) 7.28-7.36 (m, 2 H) 7.37-7.45 (m, 2H) 7.51-7.65 (m, 2H) 7.72-7.84 (m, 2H)

### Synthesis of Compound 17

Fmoc-vinylglycine 16 (1.48 g, 4.59 mmol) was dissolved in a tert-BuOH/H₂O 1:1 mixture (30 mL) and after that AD-mixβ (6.88 g) and K₂CO₃ (634 mg, 4.59 mmol) were added and the mixture was stirred at r.t for 24 h. Na₂S₂O₃ (3 g) was added and, after stirring for 15 min at r.t., the mixture was acidified to pH 1-2 using 6N HCl and then extracted 5 times with EtOAc. The solvent was removed at reduced pressure and the residue was purified by flash-column chromatography (RP-C18AQ, H₂O/ACN gradient elution from 1:2 to 1:1) giving 1.03 g of the diol derivative 17 (63%); ¹H NMR (400 MHz, acetone) d ppm 3.52-3.64 (m, 2 H) 3.67-3.82 (m, 1H) 4.20-4.51 (m, 5H) 4.52-4.63 (m, 1H) 6.23-6.42 (m, 1H) 7.29-7.36 (m, 2H) 7.37-7.45 (m, 2H) 7.69-7.79 (m, 2H) 7.82-7.89 (m, 2H).

### Synthesis of Compound 18

Compound 14 (1.02 g, 2.85 mmol) was dissolved in dry EtOAc (20 mL) and 2,2-dimethoxypropane (34 mL, 276.5 mmol). p-Toluenesulfonic acid (54.2 mg, 0.285 mmol) was added and the mixture was stirred at r.t. for 4 days. The reaction was stopped by addition of 20 ml of 5% NaHCO₃ solution and the organic solvents were then evaporated at reduced pressure. The aqueous phase was directly injected for chromatographic separation (RP-C18AQ, H₂O/ACN gradient elution from 9:1 to 7:3). The fractions containing the product were pooled and the organic solvent was removed at reduced pressure. After cooling the remaining aqueous phase in an ice bath, the pi was adjusted to ≈1-2 using 2N HCl with precipitation of the correspondent carboxylic acid. The obtained suspension was extracted 2 times with EtOAc and the combined organic layer was washed with NaCl saturated solution, dried over anhydrous Na₂SO₄ and concentrated to dryness, giving 900 mg of product (80%). ¹H NMR (400 MHz, CHLOROFORM-d) δ ppm 1.24-1.54 (m, 6H) 3.69-3.92 (m, 1H) 4.04-4.31 (m, 2H) 4.32-4.54 (m, 3H) 4.54-4.71 (m, 1H) 5.37-5.53 (m, 1H) 7.26-7.34 (m, 2H) 7.34-7.45 (m, 2H) 7.48-7.64 (m, 2H) 7.75 (d, J=7.5 Hz, 2H).

### Example 4

### Peptide Synthesis

Peptides were synthesized by means of fluorenylmethyloxycarbonyl (Fmoc) solid phase peptide synthesis on a multiple peptide synthesizer e.g. from Multisyntech. For this 4.0 equivalents of each amino acid derivative were used. Amino acid derivatives were dissolved in N-methylpyrrolidone containing 1 equivalent of 1-Hydroxy-7-azabenzotriazol. Peptides were synthesized on Tentagel R resin. Coupling reactions were carried out for 5 minutes in dimethylformamide as a reaction medium with 4 equivalents HATU and 8 equivalents of N,N-Diisopropylethylamine (DIPEA) relative to resin loading. The Fmoc group was cleaved in 8 minutes after each synthesis step using 25% piperidine in dimethyl formamide. The resin was treated with a solution of 2% hydrazine in DMF for 2×30 min to liberate ivDde-protected lysine. Afterwards 6-Maleimidohexanoic acid N-hydroxysuccinimide ester (10 eq.) and DIPEA (10 eq.) were added to the resin and incubated for 1 h followed by 3 washing steps with DMF Release of the peptide from the synthesis resin and the cleavage of the acid-labile protecting groups was achieved in 3 hours at room temperature with a cocktail containing trifluoroacetic acid, triisopropylsilane, and water (38:1:1). The reaction solution was subsequently mixed with cooled diisopropyl ether to precipitate the peptide. The precipitate was filtered, washed again with cold diisopropyl ether, dissolved in a small amount of aqueous acetic acid and lyophilized. The crude material obtained was purified by preparative RP-HPLC using a gradient of acetonitrile/water containing 0.1% trifluoroacetic acid.

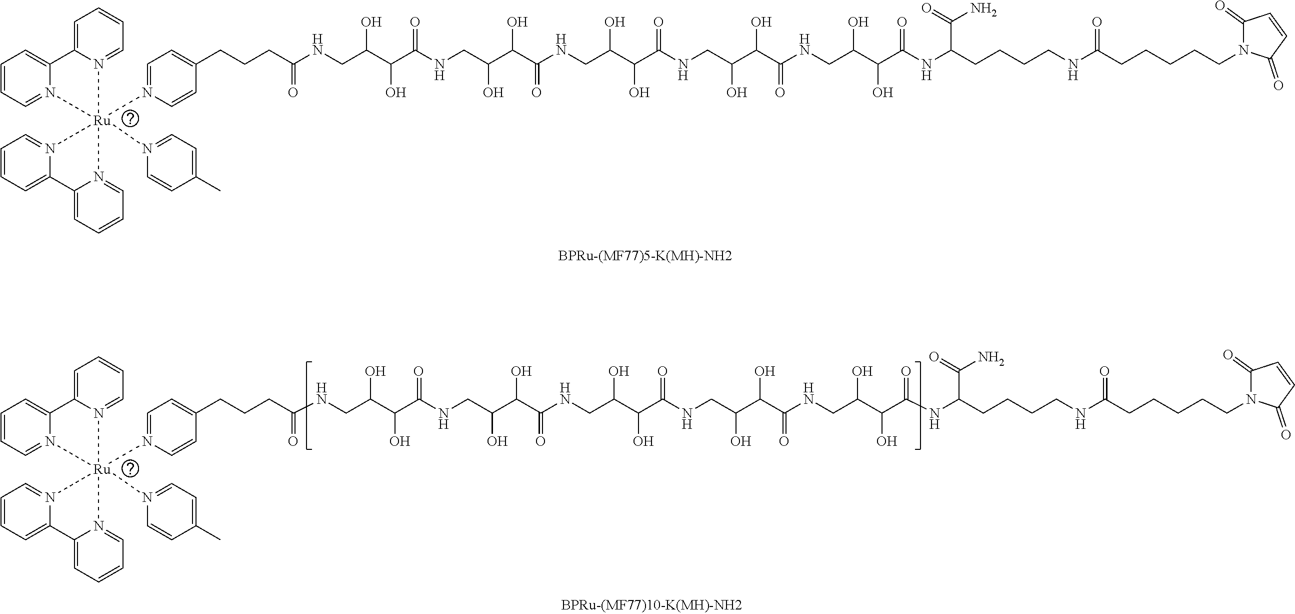

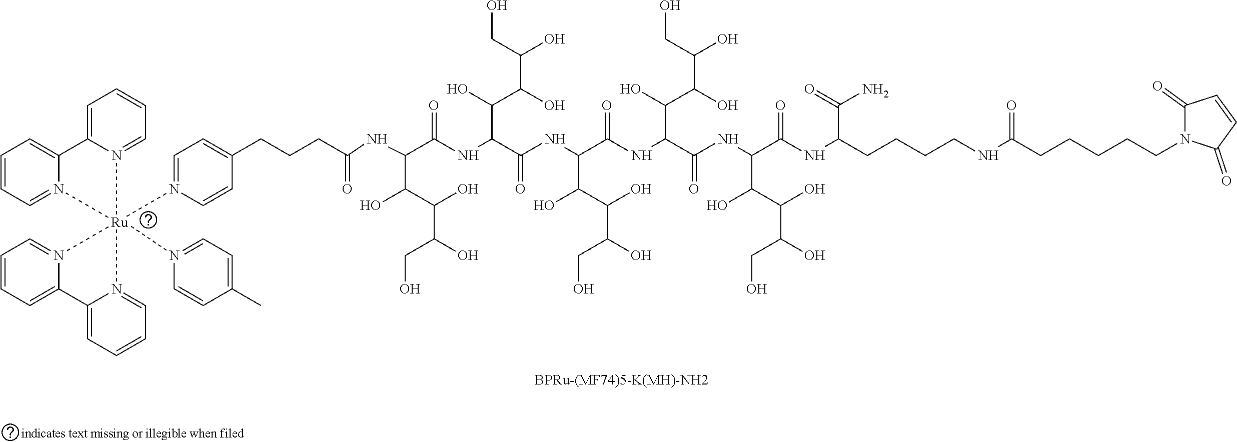

The identity of the purified material was checked by means of ion spray mass spectrometry.

BPRu-(MF74)5-K(MH)amid
Sequence: BPRu-MF74-MF74-MF74-MF74-MF74-Lys(MH)—NH2
Special amino acid derivatives:
BPRu: Ruthenium(bipyridyl)3 carboxylic acid
Fmoc-Lys(ivDde)
ESI-MS_{calc}: M⁺=1876 Da; ESI-MSₑₓₚ: [M+2H]²⁺=937 Da

BPRu-(MF77)5-K(MH)amid
Sequence: BPRu-MF77-MF77-MF77-MF77-MF77-Lys(MH)—NH2
Special amino acid derivatives:
BPRu: Ruthenium(bipyridyl)3 carboxylic acid
Fmoc-Lys(ivDde)
ESI-MS_{calc}: M+=1576 Da; ESI-MSₑₓₚ: [M+2H]²⁺=789 Da

BPRu-(MF77)10-K(MH)amid
Sequence: BPRu-MF77-MF77-MF77-MF77-MF77-MF77-MF77-MF77-MF77-MF77-Lys(MH)—NH2
Special amino acid derivatives:
BPRu: Ruthenium(bipyridyl)3 carboxylic acid
Fmoc-Lys(ivDde)
ESI-MS_{calc}: M+=2161 Da; ESI-MSₑₓₚ: [M+3H]³⁺=721 Da

BPRu-(S779)5-K(MH)amid
Sequence: BPRu-S779-S779-S779-S779-S779-Lys(MH)—NH2
Special amino acid derivatives:
BPRu: Ruthenium(bipyridyl)3 carboxylic acid
Fmoc-Lys(ivDde)
ESI-MS_{calc}: M+=1876 Da; ESI-MSₑₓₚ: [M+2H]²⁺=939 Da

BPRu-(FA36)5-K(MH)amid
Sequence: BPRu-FA36-FA36-FA36-FA36-FA36-Lys(MH)—NH2
Special amino acid derivatives:
BPRu: Ruthenium(bipyridyl)3 carboxylic acid
Fmoc-Lys(ivDde)
ESI-MS_{calc}: M+=1576 Da; ESI-MSₑₓₚ: [M+2H]²⁺=788 Da

BPRu-(FA36)5-K(MH)amid
Sequence: BPRu-FA36-FA36-FA36-FA36-FA36-FA36-FA36-FA36-FA36-FA36-Lys(MI)—NH2
Special amino acid derivatives:
BPRu: Ruthenium(bipyridyl)3 carboxylic acid
Fmoc-Lys(ivDde)
ESI-MS_{calc}: M+=1935 Da; ESI-MSₑₓₚ: [M+2H]²⁺=968 Da
Abbreviations:
DMF: Dimethylformamide
MH: Maleimidohexanoyl
BPRu-(MF74)5-K(MH)amid and BPRu-(MF74)5-K(MH)—NH2 can be used here and in the content of this disclosure interchangeably. BPRu-(MF77)5-K(MH)amid and BPRu-(MF77)5-K(MH)—NH2 can be used here and in the content of this disclosure interchangeably. BPRu-(MF77)10-K(MH)amid and BPRu-(MF77)10-K(MH)—NH2 can be used here and in the content of this disclosure interchangeably.

### General Protocol for Ir Linker-MF77 Conjugation

Step 1, Ir coupling:

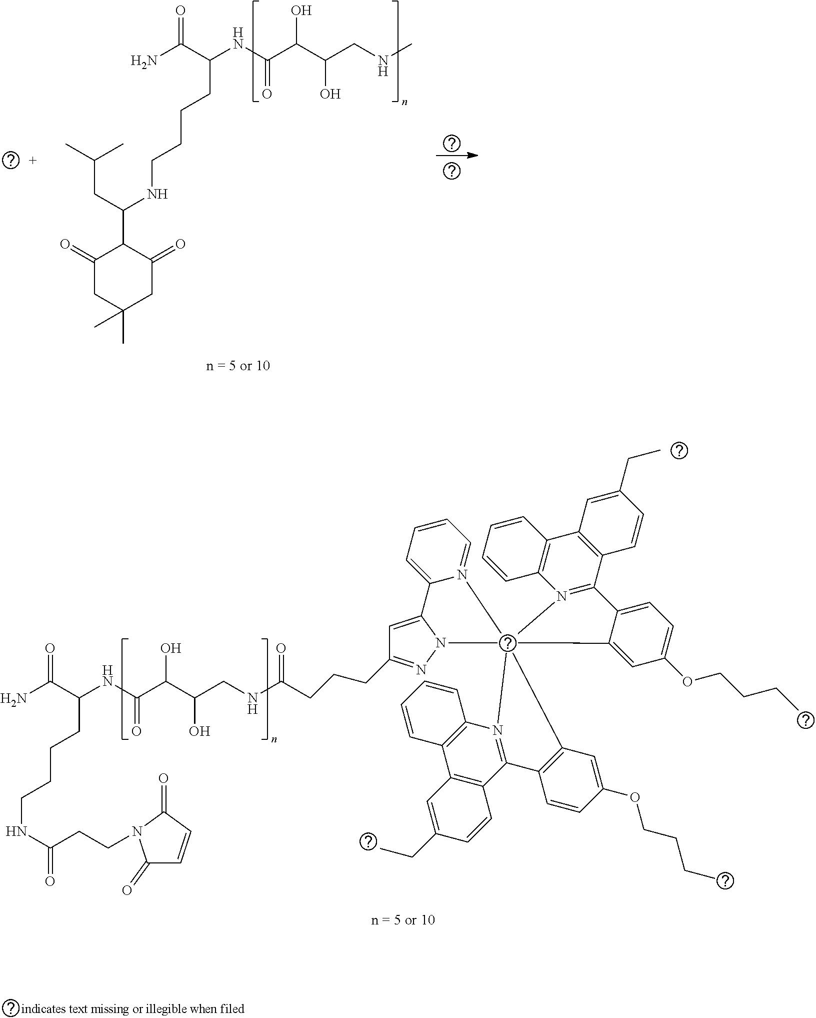

In 10 mL flash the polyol linker N^{o} (n=10, 6.6 mg, 4.34 μmol) was dissolved in 1 ml, of dry DMF: the DIPEA (2.8 ma, 21.7 μmol) and Ir3+—NHS ester (Cs+ salt, 10.5 mg, 5.20 μmol, dissolved in 1 mL of DMF) were added. The reaction was left to react at r.t. 4 h. After that, the solvent was evaporated and the red solid was dissolved in 2 mL, of H₂O and the product was purified by HPLC-prep C18, 10 mL/min, 1 injections:
Method:
0 min: 98% H₂O, 2% CH₃CN;
0-10 min: 98% H₂O, 2% CH₃CN;
10-60 min: 70% H₂O; 30% CH₃CN;
60-90 min: 20% H₂O 80% CH₃CN;

Step 2, deprotection:

The combined fractions 8.8 mg were dissolved in 4 mL of a solution 5% hydrazine in DMF and the reaction was left to react at r.t. for 2 h. After that, the solvent was evaporated and the red solid was dissolved in 2 mL of H₂O and 10 eq, of Cs₂CO₃ were added. The product was purified by HPLC-prep C18, to give 7.4 mg of product.

Step 3, maleimide coupling:

In 10 mL flash the product of step 2 (7.4 mg, 2.81 μmol) was dissolved in 2 mL of dry DMF, the DIPEA (1.8 mg, 14.04 μmol) and Maleimide-NHS ester(7.5 mg, 28.07 μmol) were added. The reaction was left to react at r.t. for 4 h. After that, the red solution was dry and the red solid was dissolved in 0.2 mL of CHCl₃; transferred in an Eppendorf and precipitated with 0.8 of E20, the solid was separated by centrifugation. The solid was purified by washing for 4 times with same method; dissolving in 0.2 mL of CHCl₃ and precipitated with 0.8 of Et₂O. After that, the solid was dry on vacuum to give 4.9 mg of red solid product N^{o}.

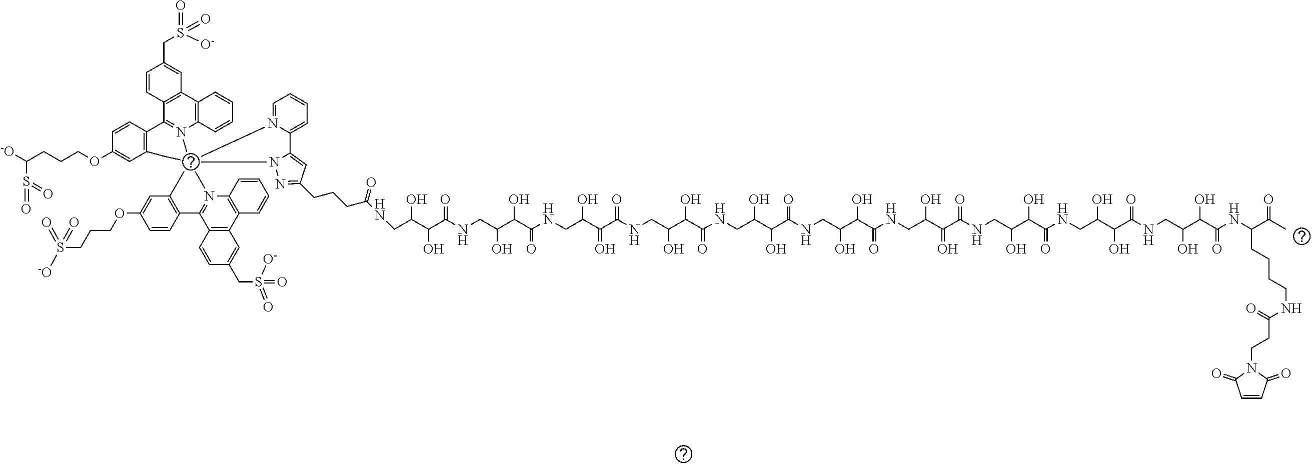

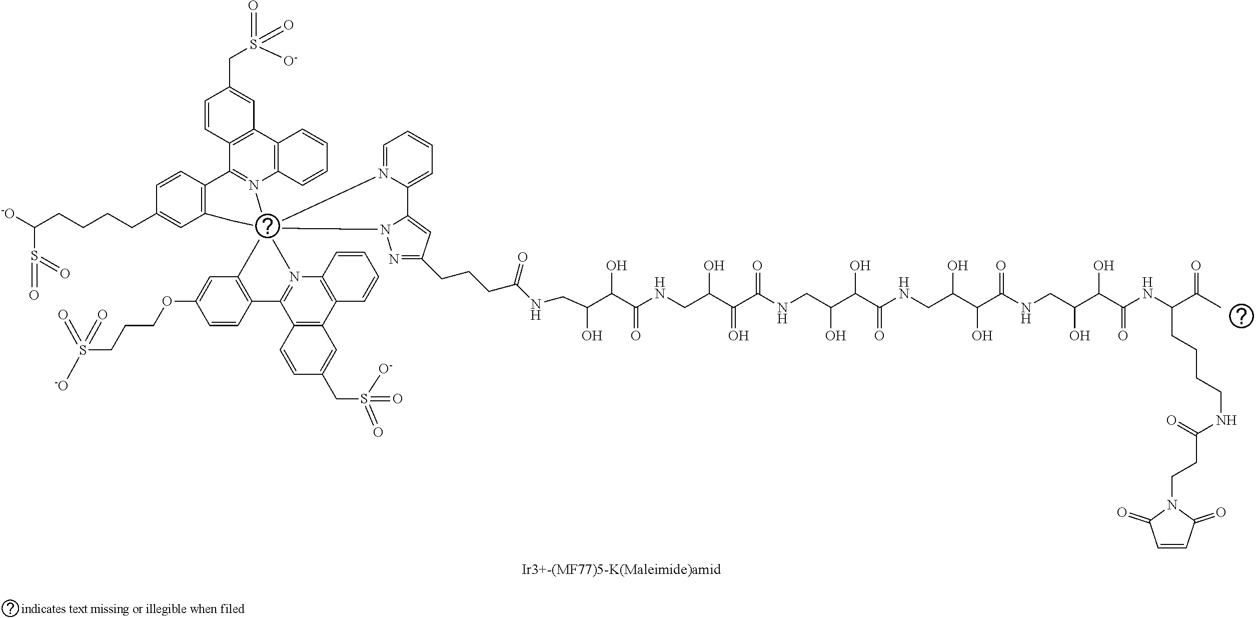

Ir3+-(MF77)10-K(Maleimide)amid
Sequence: Ir3+-MF77-MF77-MF77-MF77-MF77-MF77-MF77-MF77-MF77-MF77-Lys(Maleimide)—NH2
ESI-MS_{calc}: M+=2844 Da; ESI-MSₑₓₚ: [M+2H]²⁺=1423 Da
Polyol precursor: (MF77)10-K(ivDde)amid
Sequence: MF77-MF77-MF77-MF77-MF77-K(ivDde)-NH2
ESI-MS_{calc}: M+=1522 Da; ESI-MSₑₓₚ: [M+2H]²⁺=762 Da

Ir3+-(MF77)5-K(Maleimide)amid
Sequence: Ir3+-MF77-MF77-MF77-MF77-MF77-Lys(Maleimide)-NH2
ESI-MS_{calc}: M+=2259 Da; ESI-MSₑₓₚ: [M+2H]²⁺=1130 Da
Polyol precursor: (MF77)5-K(ivDde)amid
Sequence: MF77-MF77-MF77-MF77-MF77-K(ivDde)-NH2
ESI-MS_{calc}: M+=937 Da; ESI-MSₑₓₚ: [M+2H]²⁺=469 Da

### Example 6

Site Specific Antibody Conjugates

ThioMab Variants from the Elecsys anti-Troponin T clone 5D8 were conjugated with different Ruthenium labels (with different linkers) at position A114C and S374C; respectively, according to the protocol described in Bhakta S. et al., 2013 (Engineering THIOMABs for site-specific conjugation of thiol-reactive linkers, Methods Mol Biol) These conjugates were then used to run the Roche Troponin T hs Elecsys assay (Id. 05092744190, Roche Diagnostics GmbH, Mannheim, Germany) replacing the original R2 reagent (detection reagent) at different concentrations.

Non-site specific conjugation of labels (SATP-Maleimide)

The Elecsys high sensitive Troponin-T (HS Tn-T) clone 5D8 used for conjugation and ELC (electro-chemoluminescence) performance evaluation of the newly synthesized Maleimido-Ru (Ruthenium) complexes containing diverse polyol linkers. To generate functionalized antibody, Thiol functionality was introduced to the IgG by conjugating with N-Succinimidyl-S-acetylthiopropionate (SATP) at a stoichiometry of 1:5 (IgG:SATP) to generate MAB<Tn-T>chim-5D8-IgG-SATP(1:5). The acetyl protection from sulfur was removed by hydroxylamine treatment to get the final sulfhydryl-containing antibody. This SH-antibody was then were conjugated with maleimodo-polyol-Ruthenium labels in 50 mM KPP. 150 mM KCl, pH 7.4 with 5% DMSO.

### Example 7

Elecsys Performance

All assays variants were run on a Cobas E170 Module using the Troponin T hs assay protocol with a blank control (Diluent Multiassay, Id. 11732277122, Roche Diagnostics GmbH, Mannheim, Germany, Cal1 and Cal2 from Troponin T hs CalSet (Id. 05092752190, Roche Diagnostics GmbH, Mannheim, Germany) using the Troponin T assay specifications. Incorporation rate of PEG and polyol based linkers are similar (see Incorp rate). The newly polyol linker FA41 shows comparable signal to noise ratio in low (Cal1/MA) and high range (Cal2/MA) of TnT analyte, with significantly improved linker stability over last generation polyol (MF74). MA: serum blank; Cal1: 18 ng/L; Cal2: 4200 ng/L.

These conjugates were then used in Troponin T hs Elecsys assay variants (Id. 05092744190, Roche Diagnostics GmbH, Mannheim, Germany) replacing the original R2 reagent at 2.5 μg/ml concentration. First ECL measurement was done after one week incubation of the conjugated at 4° C. TnT R2 buffer

TABLE 1
Elecsys performance of Non-site specific conjugation.

| | Incorp. | | | | | |
|---|---|---|---|---|---|---|
| Label name | Rate | MADil | Cal1 | Cal2 | Cal1/MA | Cal2/MA |
| BPRu-MEA | 2.5 | 869 | 1167 | 262500 | 1.3 | 302 |
| BPRu-PEG23- | 3.3 | 951 | 1152 | 255800 | 1.2 | 269 |
| MH | | | | | | |
| BPRu-(MF74)₅- | 5.9 | 852 | 1629 | 440350 | 1.9 | 517 |
| K(MH)amid | | | | | | |
| BPRu-(MF77)5- | 2.3 | 1519 | 1245 | 290971 | 0.8 | 192 |
| K(MH)amid | | | | | | |
| BPRu- | 2.4 | 824 | 1033 | 230400 | 1.3 | 280 |
| (MF77)₁₀- | | | | | | |
| K(MH)amid | | | | | | |
| BPRu-(S779)5- | 2.5 | 10030 | 2519 | 214600 | 0.3 | 21 |
| K(MH)amid | | | | | | |

Branched polyol linker (MF74 based) gives better signal to noise (Cal1/MA and Cal2/MA) compared to PEG23 and no linker (BPRu-MEA).

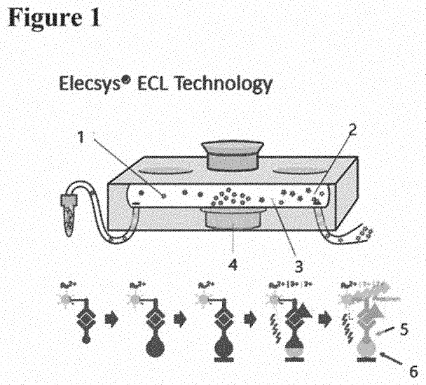

FIG. 1 shows the principle of an Elecsys ECL technology and the function of a possible Elecsys antibody test. ECL (ElectroChemiLuminescence, 1—magnetizable microparticles with bound antigen-antibody complex, 2—unbounded conjugate, 3—flow channel, 4—magnet, 5—e.g. biotinylated antibody, 6—e.g. streptavidin-coated magnetic beads) is Roche's technology for immunoassay detection. Based on this technology and combined with well-designed, specific and sensitive immunoassays, Elecsys delivers reliable results. The development of ECL immunoassays can be based on the use of a ruthenium-complex and tripropylamine (TPA). The chemiluminescence reaction for the detection of the reaction complex is initiated by applying a voltage to the sample solution resulting in a precisely controlled reaction. ECL technology can accommodate many immunoassay principles while providing superior performance.

Table 2 shows that BPRu-(MF77)₅K(MH)amide, BPRu-(S779)₅-MH and BPRu-(MF74)₅K(MH)amide have similar retention times of 6.85, 6.75, and 6.72 min in RP HPLC (water/acetonitrile+0.1% TFA). The corresponding PEG linked compound has a retention time of 9.73 min.

TABLE 2
Retention time of BPRu-(MF77)₅K(MH)amide, BPRu-(S779)5-
MH, BPRu-(MF74)₅K(MH)amide and BPRu-PEG24-MH

| Label Name | Retention Time |
|---|---|
| BPRu-PEG24-MH | 9.73 min |
| BPRu-(MF74)5-MH | 6.72 min |
| BPRu-(MF77)5-MH | 6.85 min |
| BPRu-(S779)5-MH | 6.75 min |

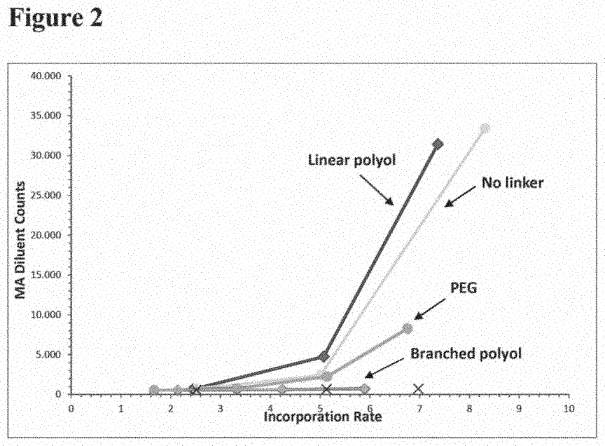

FIG. 2 shows results of the Elecsys E170: Troponin T hs Assay (Blank Values) by using the method of the present invention and by using comparative methods with PEG-linker and no linker, respectively. It is shown the Multiassay (MA) Diluent Counts as a function of the incorporation rate. Incorporation rate means here the average number of ECL labels per antibody. The Multiassay Diluent comprises a blank and 2.5 μg/mL conjugate. The term conjugate and complex can be used interchangeable in the whole disclosure. The conjugation procedure is described e.g. in example 6.

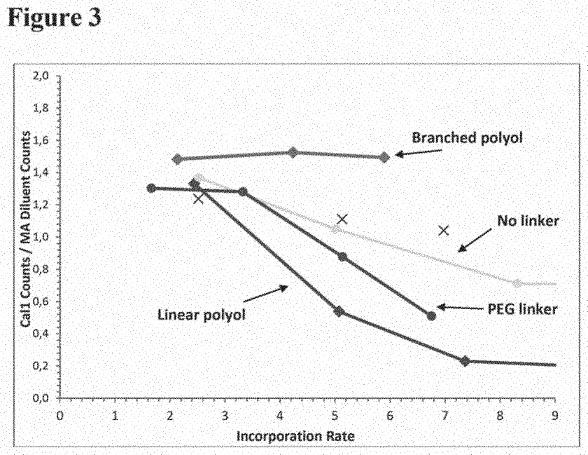

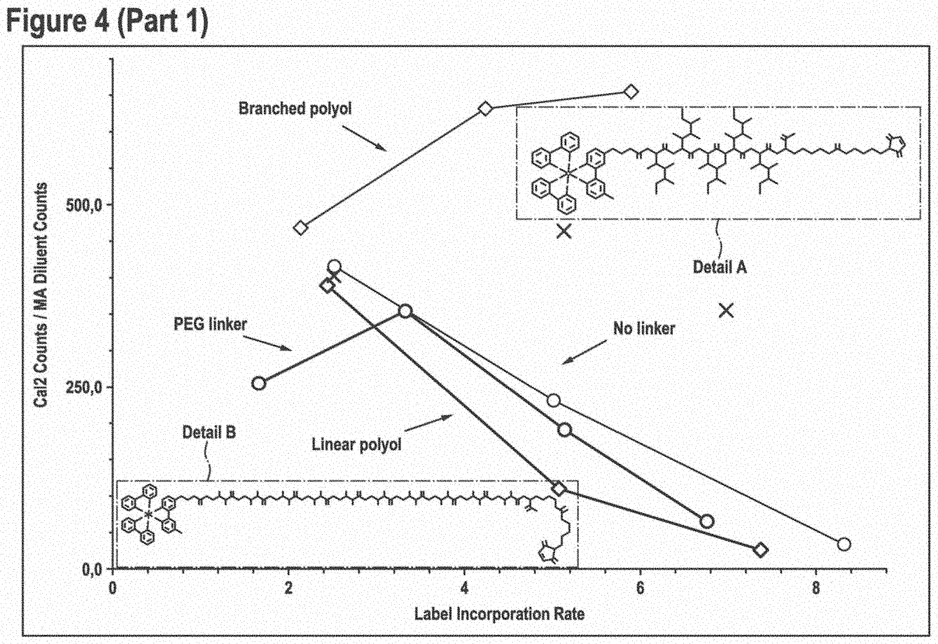

FIGS. 3 and 4 show results of the Elecsys E170: Troponin T hs Assay by using the method of the present invention and by using comparative methods with PEG-linker and no linker, respectively. It is shown the Cal1 Counts/Multiassay (MA) Diluent Counts and Cal2 Counts/Multiassay (MA) Diluent Counts as a function of the label incorporation rate. Label incorporation rate means here the average number of ECL labels per antibody. The Cal1/Multiassay Diluent and Cal2/Multiassay Diluent comprises no prewash step, and 2.5 μg/mL conjugate.

This patent application claims the priority of the European patent application 20216267.3, wherein the content of this European patent application is hereby incorporated by references.

1. A method for detecting an analyte of interest in a sample comprising:
a) providing the sample comprising the analyte of interest,
b) providing a complex comprising a linker, wherein the linker covalently binds a label compound and an analyte-specific binding agent, wherein the label compound is capable of producing a detectable signal,
c) coupling the sample of step a) with the complex of step b), and
d) detecting the analyte of interest by using the detectable signal of the label compound,
wherein the complex is a compound of formula I: 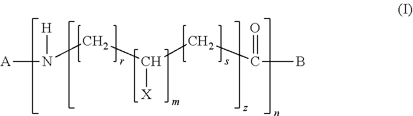
wherein A represents the label compound and B represents the analyte-specific binding agent or vice versa,
X is OH or (CHOH)ₜ—CH₂OH with t≥1,
m is an integer from 1 to 8,
n is an integer from 2 to 20,
r is an integer and ≥0, wherein r≥1 in case X=OH,
s is an integer and ≥0, and
z is an integer and ≥1.

2. The method of claim 1, wherein the complex is a compound of formula II: 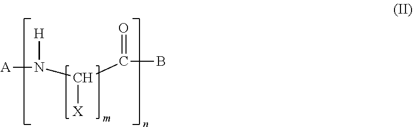
wherein each of A, B, m, and n has the same meaning as mentioned in claim 1,
wherein X is (CHOH)ₜ—CH₂OH with t≥1.

3. The method of claim 1, wherein the complex is a compound of formula III: 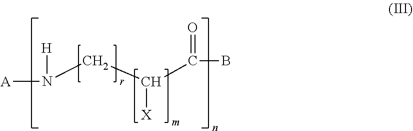
wherein each of A, B, m, and n has the same meaning as mentioned in claim 1,
wherein X is OH and r≥1.

4. The method of claim 1, wherein X is OH and/or m is 2 or 4 or 6.

5. The method of claim 1, wherein X is (CHOH)ₜ—CH₂OH with t=1, 3, 5, or 7.

6. The method of claim 1, wherein the label compound is covalently bonded to the linker via a first conjugation method, wherein the first conjugation method is selected from the group consisting of click chemistry, amide, ester, imide, carbonate, carbamate, squarate, thiazole, thiazolidine, hydrazone, oxime, dihydropyridazine, thiol-maleimide, cycloaddition, photoclick, Staudinger ligation, Diels-Alder, tetrazine ligation, cross-coupling, Pictet-Spengler, and quadricylcane,
and/or
wherein the analyte-specific binding agent is selected from the group consisting of an antibody, an analyte-specific fragment and/or derivative of an antibody, an aptamer, a spiegelmer, a darpin, a lectin, an ankyrin repeat containing protein, and a Kunitz type domain containing protein.

7. The method of claim 1, wherein the complex is a compound of formula IV-1: 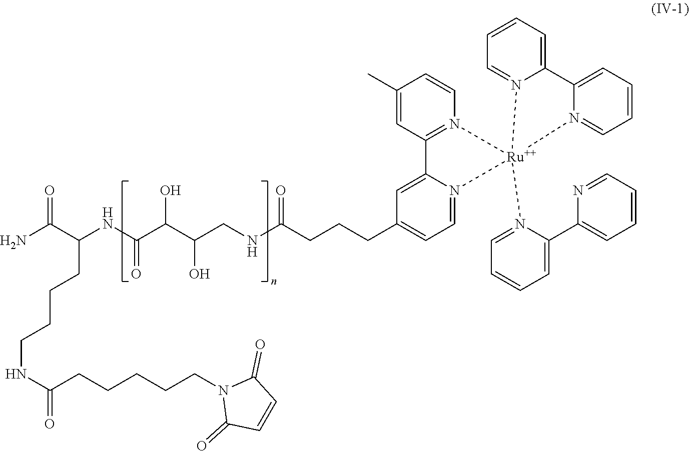
wherein n is more than 1.

8. The method of claim 1, wherein the complex is a compound of formula V or VI: 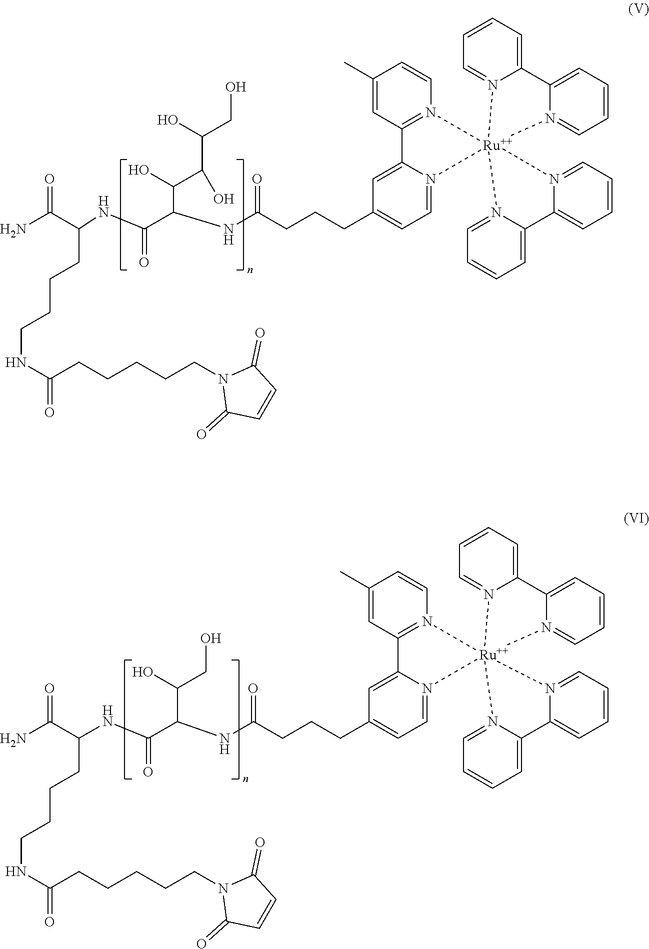
wherein n of formula V or VI is independently of each other more than 1.

9. The method of claim 1, wherein step b) comprises a peptide-based synthesis.

10. (canceled)

11. A kit for performing detection of an analyte of interest in a sample, the kit comprising in separate containers
a) a solid phase capable of immobilizing the analyte; and
b) a compound of formula I: 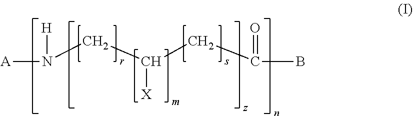
wherein A represents the label compound and B represents the analyte-specific binding agent or vice versa,
X is OH or (CHOH)ₜ—CH₂OH with t≥1,
m is an integer from 1 to 8,
n is an integer from 2 to 20,
r is an integer and ≥0, wherein r≥1 in case X=OH,
s is an integer and ≥0, and
z is an integer and ≥1.

12. (canceled)

13. A complex of formula I, 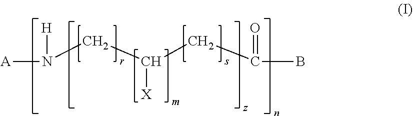
wherein A represents the label compound and B represents the analyte-specific binding agent or vice versa,
X is OH or (CHOH)ₜ—CH₂OH with t≥1,
m is an integer from 1 to 8,
n is an integer from 2 to 20,
r is an integer and ≥0, wherein r≥1 in case X=OH,
s is an integer and ≥0, and
z is an integer and ≥1.

14. A method to synthesize a complex of claim 13 comprising:
a) providing a monomer or derivatives thereof, wherein the monomer is an amino acid comprising an amino group, a carboxy group, and at least one hydroxyl group, wherein the amino group or the carboxy group is protected by a first protecting group, and the at least one or each hydroxyl group is protected by a second protecting group, and
b) using the monomer in a process of solid phase peptide synthesis, cleaving the first and second protection group and forming a complex of formula III, 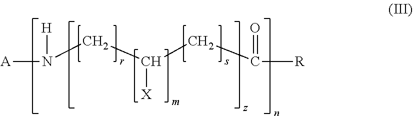
wherein A represents the label compound and R represents a second spacer or vice versa,
wherein R is capable of covalently bonding to an analyte-specific binding agent or is covalently bonded to an analyte-specific binding agent,
X is OH or (CHOH)ₜ—CH₂OH with t≥1,
m is an integer from 1 to 8,
n is an integer from 2 to 20,
r is an integer and ≥0, wherein r≥1 in case X=OH,
s is an integer and ≥0, and
z is an integer and ≥1.

15. The method of claim 14, wherein a monomer or a derivative thereof is selected from the following formulae m-1 to m-4: 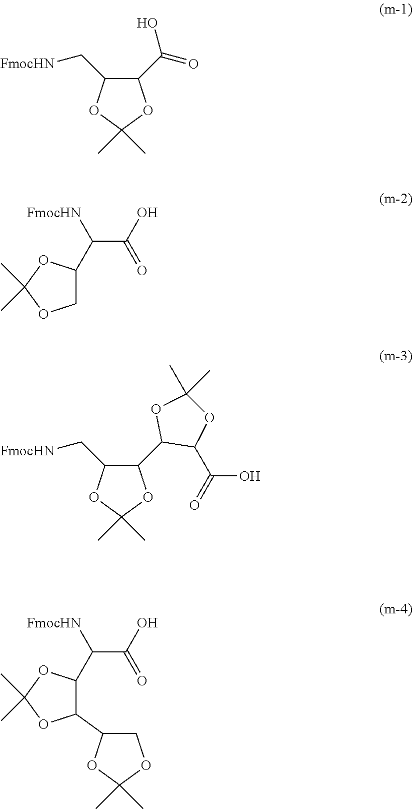
wherein FmocHN means an amine protected with a 9-fluorenylmethoxycarbonyl protecting group.

16. The method of claim 1, wherein the detectable signal is a chemiluminescence based signal.

17. The method of claim 9, wherein the peptide-based synthesis is a solid phase peptide synthesis (SPPS).

18. The method of claim 13, wherein the compound is suitable to detect an analyte of interest in a sample.

19. The method of claim 1, wherein:
X is OH or (CHOH)ₜ—CH₂OH with t=1, 3, 5 or 7,
m is an integer and ≥2,
n is an integer from 5 to 20,
r is an integer and 0, and
s is an integer and 0.

20. The kit of claim 11, wherein:
X is OH or (CHOH)ₜ—CH₂OH with t=1, 3, 5 or 7,
m is an integer and ≥2,
n is an integer from 5 to 20,
r is an integer and 0, and
s is an integer and 0.

21. The complex of claim 13, wherein:
X is OH or (CHOH)ₜ—CH₂OH with t=1, 3, 5 or 7,
m is an integer and ≥2,
n is an integer from 5 to 20,
r is an integer and 0, and
s is an integer and 0.

22. The method of claim 14, wherein:
X is OH or (CHOH)ₜ—CH₂OH with t=1, 3, 5 or 7,
m is an integer and ≥2,
n is an integer from 5 to 20,
r is an integer and 0, and
s is an integer and 0.
